(19) United States
(12) Patent Application Publication
(10) Pub. No.: US 2024/0327759 A1
(43) Pub. Date: Oct. 3, 2024

(54) MULTI-STEP METHODS OF CLEANING DAIRY MEMBRANES

(71) Applicant: ECOLAB USA INC., Saint Paul, MN (US)

(72) Inventors: Stephan Doerries, Düsseldorf (DE); Matthias Schmitz, Düsseldorf (DE); Ralf Krack, Hilden (DE); Paige Mary Owens-Polta, Saint Paul, MN (US); Krista Otting, Saint Paul, MN (US); Sukhwan Soontravanich, Saint Paul, MN (US); Jesse Ray Murphy, Saint Paul, MN (US)

(21) Appl. No.: 18/621,678

(22) Filed: Mar. 29, 2024

Related U.S. Application Data
(60) Provisional application No. 63/492,942, filed on Mar. 29, 2023.

(51) Int. Cl. C11D 3/386; C11D 1/14; C11D 1/22; C11D 1/66; C11D 1/72; C11D 1/75; C11D 3/04; C11D 3/10; C12N 9/20

(52) U.S. Cl. CPC C11D 3/38627; C11D 1/143; C11D 1/146; C11D 1/22; C11D 1/662; C11D 1/72; C11D 1/75; C11D 3/044; C11D 3/10; C11D 3/38618; C11D 3/38663; C12N 9/20; C12Y 301/01003

(57) ABSTRACT

The present disclosure relates generally to the field of dairy membrane cleaning. In particular, the methods comprise stepwise treatment of the dairy membranes to beneficially remove fouling and clean the membranes. The methods are suitable for dairy membranes including microfiltration, ultrafiltration, nanofiltration, and reverse osmosis membranes utilized in dairy production.

### TECHNICAL FIELD

The present disclosure relates generally to the field of dairy membrane cleaning. In particular, the methods comprise stepwise treatment of the dairy membranes to beneficially remove fouling and clean the membranes. The methods are suitable for dairy membranes including microfiltration, ultrafiltration, nanofiltration, and reverse osmosis membranes utilized in dairy production.

### BACKGROUND

A critical challenge in dairy production is fat fouling in filtration membranes. Studies of cleaned and uncleaned field membranes showed a factor of 10 times higher fouling load of fats, especially high-melting triglycerides compared to protein foulants present on the membranes of globally sampled dairy membranes. Fat and protein soils are difficult to clean using a standard cleaned-in-place regime. Further, due to the higher melting points of the fat, far beyond the cleaned-in place temperature, high melting fat accumulates over the membrane's lifetime. Incomplete removal of membrane fouling impacts membrane production performance and is a significant factor in overall membrane health. Triglyceride fats are challenging to remove and build up on the membrane over time. Triglyceride fats can be degraded via a catalytic reaction with lipase. The lipase molecule separates the fatty acid moiety from the glycerin proportion.

Lipases, although able to remove fats from the membrane, can also stick to the membrane surface and pores depending on the size of the membrane. Lipase molecules could have sizes of around d30 kDa, which means that based purely on the size of the molecule, it will pass through or permeate most microfiltration membranes, whereas it would be rejected by ultrafiltration, nanofiltration, and reverse osmosis membranes. If lipase can enter the structure of a microfiltration membrane, the larger internal pore area interacts with the lipase causing more lipase molecules sticking to the membrane, which also makes a rinse out more difficult. For porous membranes, lipase could interact with the pores on the membrane surface. As the membrane surface becomes smoother (i.e., smaller pore size) as more the lipase interaction with the membranes decreases.

Current cleaning regimes do not completely remove all the fouling components from all membrane types and do not address the full fouling phenomena. Lipases are surface active and stick to the membrane surface and cannot be rinsed away with water. Lipases cannot be inactivated with acids or alkaline. Oxidizers likewise cannot fully inactivate lipase (stability against oxidation). Thus, a need for dairy membrane cleaning composition and methods to clean dairy membranes are needed to overcome these downfalls of current clean-in-place dairy membrane cleaning regimes.

### BRIEF SUMMARY OF PREFERRED EMBODIMENTS

Disclosed herein are methods of cleaning dairy membranes. These methods provide various advantages over existing dairy membrane cleaning methodologies. For example, the methods provide a mechanism to retain enzyme activity during the enzyme cleaning steps and then to deactivate the lipase to avoid degradation of the produced dairy product due to still active lipase in the production process, which creates an “off-taste” of dairy products via creation of small chain fatty acids. Another advantage of the methods is that they are suitable for a variety of types of dairy filtration membranes, including, microfiltration, ultrafiltration, nanofiltration, and reverse osmosis membranes and others. Other advantages and benefits of the disclosed methods are described herein.

A preferred embodiment comprises a method of cleaning a dairy filtration membrane comprising contacting the dairy filtration membrane with an enzyme composition, wherein the enzyme composition comprises a lipase and a buffer, wherein the pH of the enzyme composition is from about 7.5 to about 11.0; contacting the dairy filtration membrane with a surfactant composition, wherein the surfactant composition comprises an alkyl polyglucoside, an alkyl polypentoside, an amine oxide, an alcohol ethoxylate, an alkoxylated block copolymer, a sulfonated surfactant, or a mixture thereof; wherein the pH of the surfactant composition is from about 7.5 to about 11.0; rinsing the dairy filtration membrane; contacting the dairy filtration membrane with an acidic composition, wherein the acidic composition comprises an acid, an acidic anionic surfactant, or a mixture thereof; wherein the pH of the acidic composition is about 2.5 or lower; wherein the acidic composition deactivates the lipase; and rinsing the dairy filtration membrane.

A preferred embodiment comprises a method of cleaning a dairy filtration membrane comprising contacting the dairy filtration membrane with an enzyme composition, wherein the enzyme composition comprises a lipase, a protease, and a buffer, wherein the pH of the enzyme composition is from about 8.0 to about 10.5; wherein the dairy filtration membrane comprises a microfiltration membrane, an ultrafiltration membrane, nanofiltration membrane, and/or a reverse osmosis membrane; contacting the dairy filtration membrane with a surfactant composition, wherein the surfactant composition comprises an alkyl polyglucoside, an alkyl polypentoside, an amine oxide, an alcohol ethoxylate, an alkoxylated block copolymer, a sulfonated surfactant, or a mixture thereof; wherein the pH of the surfactant composition is from about 8.0 to about 10.5; rinsing the dairy filtration membrane; contacting the dairy filtration membrane with an acidic composition, wherein the acidic composition comprises a linear alkyl sulfonate, a branched alkyl sulfonate, a substituted aromatic sulfonate, an unsubstituted aromatic sulfonate, an organic acid, nitric acid, phosphoric acid, methanesulfonic acid, or mixture thereof, or a mixture thereof; wherein the pH of the acidic composition is about 2.5 or lower; wherein the acidic composition deactivates the lipase; and rinsing the dairy filtration membrane.

In a more preferred embodiment, the method can further comprise contacting the dairy filtration membrane with an alkaline composition; wherein the alkaline composition comprises an alkali metal hydroxide, alkali metal carbonate, an alkali metal silicate, an organic alkalinity source, or mixture thereof; wherein the alkaline composition has a pH from about 10.0 to about 12.0; and rinsing the dairy filtration membrane.

### BRIEF DESCRIPTION OF THE DRAWINGS

FIG. 1 is a flow diagram of the exemplary method of cleaning dairy membranes.

FIG. 2 illustrates lipase interaction in an exemplary membrane.

FIG. 3A illustrates lipase in a liquid solution.

FIG. 3B illustrates lipase within an exemplary membrane.

FIG. 3C shows a scanning electron microscope (SEM) image of lipase within an exemplary membrane.

FIG. 4 is a table showing lipase inactivation by standard cleaning regimes.

FIG. 5 is a table comparing lipase inactivation by standard cleaning regimes and exemplary cleaning regimes.

FIG. 6 is a table showing lipase activity of microfiltration, ultrafiltration, nanofiltration, and reverse osmosis membranes.

FIG. 7 is a table showing lipase inactivation by exemplary cleaning regimes on 10 different membranes.

FIG. 8 is a table showing lipase inactivation by exemplary cleaning regimes on 3 different membranes.

Various embodiments of the present invention will be described in detail with reference to the drawings, wherein like reference numerals represent like parts throughout the several views. Reference to various embodiments does not limit the scope of the invention. Figures represented herein are not limitations to the various embodiments according to the invention and are presented for exemplary illustration of the invention.

### DETAILED DESCRIPTION

The present disclosure relates to methods of cleaning dairy membranes, including, microfiltration, ultrafiltration, nanofiltration, and reverse osmosis membranes utilized in dairy production. Beneficially, the methods employ a stepwise treatment that reduces can minimize fouling and optimize the cleaning agents.

It is further to be understood that all terminology used herein is for the purpose of describing particular embodiments only, and is not intended to be limiting in any manner or scope. For example, as used in this specification and the appended claims, the singular forms “a,” “an” and “the” can include plural referents unless the content clearly indicates otherwise. Further, all units, prefixes, and symbols may be denoted in its SI accepted form.

Numeric ranges recited within the specification are inclusive of the numbers defining the range and include each integer within the defined range. Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾. This applies regardless of the breadth of the range.

### Definitions

So that the present invention may be more readily understood, certain terms are first defined. Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation, the preferred materials and methods are described herein. In describing and claiming the embodiments of the present invention, the following terminology will be used in accordance with the definitions set out below.

The term “about,” as used herein, refers to variation in the numerical quantity that can occur, for example, through typical measuring techniques and equipment, with respect to any quantifiable variable, including, but not limited to, concentration, mass, volume, time, temperature, and pH. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations. Whether or not modified by the term “about,” the claims include equivalents to the quantities.

The methods and compositions of the present invention may comprise, consist essentially of, or consist of the components and ingredients of the present invention as well as other ingredients described herein. As used herein, “consisting essentially of” means that the methods, systems, apparatuses and compositions may include additional steps, components or ingredients, but only if the additional steps, components or ingredients do not materially alter the basic and novel characteristics of the claimed methods, systems, apparatuses, and compositions.

The methods and compositions of the present invention may comprise, consist essentially of, or consist of the components and ingredients of the present invention as well as other ingredients described herein. As used herein, “consisting essentially of” means that the methods, systems, apparatuses and compositions may include additional steps, components or ingredients, but only if the additional steps, components or ingredients do not materially alter the basic and novel characteristics of the claimed methods, systems, apparatuses, and compositions.

The term “actives” or “percent actives” or “percent by weight actives” or “actives concentration” are used interchangeably herein and refers to the concentration of those ingredients involved in cleaning expressed as a percentage minus inert ingredients such as water or salts. It is also sometimes indicated by a percentage in parentheses, for example, “chemical (10%).”

As used herein, the term “alkyl” or “alkyl groups” refers to saturated hydrocarbons having one or more carbon atoms (e.g., C1-C20), including straight-chain alkyl groups (e.g., methyl, ethyl, propyl, butyl, pentyl, hexyl, heptyl, octyl, nonyl, decyl, etc.), cyclic alkyl groups (or “cycloalkyl” or “alicyclic” or “carbocyclic” groups) (e.g., cyclopropyl, cyclopentyl, cyclohexyl, cycloheptyl, cyclooctyl, etc.), branched-chain alkyl groups (e.g., isopropyl, tert-butyl, sec-butyl, isobutyl, etc.), and alkyl-substituted alkyl groups (e.g., alkyl-substituted cycloalkyl groups and cycloalkyl-substituted alkyl groups).

Unless otherwise specified, the term “alkyl” includes both “unsubstituted alkyls” and “substituted alkyls.” As used herein, the term “substituted alkyls” refers to alkyl groups having substituents replacing one or more hydrogens on one or more carbons of the hydrocarbon backbone. Such substituents may include, for example, alkenyl, alkynyl, halogeno, hydroxyl, alkylcarbonyloxy, arylcarbonyloxy, alkoxycarbonyloxy, aryloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), imino, sulfhydryl, alkylthio, arylthio, thiocarboxylate, sulfates, alkylsulfinyl, sulfonates, sulfamoyl, sulfonamido, nitro, trifluoromethyl, cyano, azido, heterocyclic, alkylaryl, or aromatic (including heteroaromatic) groups.

In some embodiments, substituted alkyls can include a heterocyclic group. As used herein, the term “heterocyclic group” includes closed ring structures analogous to carbocyclic groups in which one or more of the carbon atoms in the ring is an element other than carbon, for example, nitrogen, sulfur or oxygen. Heterocyclic groups may be saturated or unsaturated. Exemplary heterocyclic groups include, but are not limited to, aziridine, ethylene oxide (epoxides, oxiranes), thiirane (episulfides), dioxirane, azetidine, oxetane, thietane, dioxetane, dithietane, dithiete, azolidine, pyrrolidine, pyrroline, oxolane, dihydrofuran, and furan. As used herein, the term “soil” or “stain” refers to a non-polar oily substance which may or may not contain particulate matter such as mineral clays, sand, natural mineral matter, carbon black, graphite, kaolin, environmental dust, etc.

As used herein, the term “cleaning” refers to a method used to facilitate or aid in soil removal, bleaching, microbial population reduction, and any combination thereof. As used herein, the term “microorganism” refers to any noncellular or unicellular (including colonial) organism. Microorganisms include all prokaryotes. Microorganisms include bacteria (including cyanobacteria), spores, lichens, fungi, protozoa, virinos, viroids, viruses, phages, and some algae. As used herein, the term “microbe” is synonymous with microorganism.

As used herein, the phrase “food processing surface” refers to a surface of a tool, a machine, equipment, a structure, a building, or the like that is employed as part of a food processing, preparation, or storage activity. Examples of food processing surfaces include surfaces of food processing or preparation equipment (e.g., slicing, canning, or transport equipment, including flumes), of food processing wares (e.g., utensils, dishware, wash ware, and bar glasses), and of floors, walls, or fixtures of structures in which food processing occurs. Food processing surfaces are found and employed in food anti-spoilage air circulation systems, aseptic packaging sanitizing, food refrigeration and cooler cleaners and sanitizers, ware washing sanitizing, blancher cleaning and sanitizing, food packaging materials, cutting board additives, third-sink sanitizing, beverage chillers and warmers, meat chilling or scalding waters, autodish sanitizers, sanitizing gels, cooling towers, food processing antimicrobial garment sprays, and non-to-low-aqueous food preparation lubricants, oils, and rinse additives.

The term “hard surface” refers to a solid, substantially non-flexible surface such as a counter top, tile, floor, wall, panel, window, plumbing fixture, kitchen and bathroom furniture, appliance, engine, circuit board, dish, mirror, window, monitor, touch screen, and thermostat. Hard surfaces are not limited by the material; for example, a hard surface can be glass, metal, tile, vinyl, linoleum, composite, wood, plastic, etc. Hard surfaces may include for example, health care surfaces and food processing surfaces.

As used herein, the term “instrument” refers to the various medical or dental instruments or devices that can benefit from cleaning with a composition according to the present invention.

As used herein, the term “microorganism” refers to any noncellular or unicellular (including colonial) organism. Microorganisms include all prokaryotes. Microorganisms include bacteria (including cyanobacteria), spores, lichens, fungi, protozoa, virinos, viroids, viruses, phages, and some algae. As used herein, the term “microbe” is synonymous with microorganism.

As used herein, the term “substantially free” refers to compositions completely lacking the component or having such a small amount of the component that the component does not affect the performance of the composition. The component may be present as an impurity or as a contaminant and shall be less than 0.5 wt-%. In another embodiment, the amount of the component is less than 0.1 wt-% and in yet another embodiment, the amount of component is less than 0.01 wt-%.

The terms “water soluble” and “water dispersible” as used herein, means that the ingredient is soluble or dispersible in water in the inventive compositions. In general, the ingredient should be soluble or dispersible at 25° C. concentration of between about 0.1 wt. % and about 15 wt. % of the water, more preferably at a concentration of between about 0.1 wt. % and about 10 wt. %.

The term “weight percent,” “wt. %,” “wt-%,” “percent by weight,” “% by weight,” and variations thereof, as used herein, refer to the concentration of a substance as the weight of that substance divided by the total weight of the composition and multiplied by 100.

### Methods of Cleaning Dairy Membranes

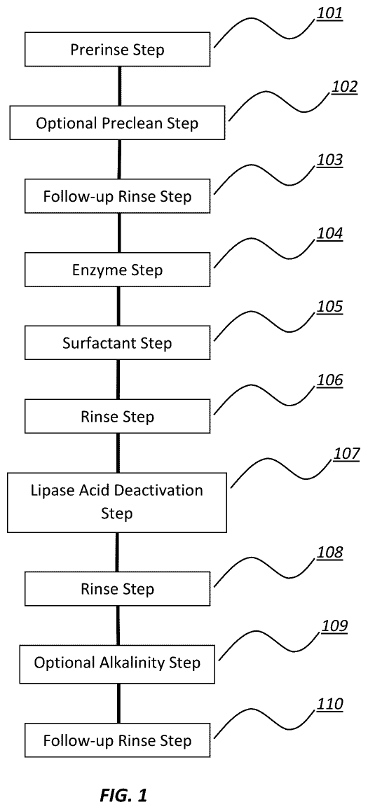

Disclosed herein are methods of cleaning dairy membranes including microfiltration, ultrafiltration, nanofiltration, or reverse osmosis or other membranes or membrane processes, typically utilized in dairy production. Preferably the dairy membranes can be cleaned with a stepwise cleaning regime employing a prerinse step 101, an optional preclean step 102, a follow-up rinse step 103, an enzyme step 104, a surfactant step 105, a rinse step 106, a lipase acid deactivation step 107, a rinse step 108, an optional alkalinity step 109, and a follow-up rinse step 110, as illustrated in FIG. 1. More preferably, the dairy membranes cleaned in the enzyme step 104 are cleaned with an enzyme composition comprising a carrier, an enzyme and a buffer. Most preferably the enzyme composition comprises water, lipase, protease, buffer, and a chelant. More preferably, the dairy membranes cleaned in the lipase acid deactivation step 107 are cleaned with an acidic composition. Most preferably, the acidic composition comprises an acid, an acidic anionic surfactant or a mixture thereof. More preferably, the dairy membranes cleaned in the alkalinity step 109 are cleaned with an alkaline composition. Most preferably, the alkaline composition comprises an alkali metal hydroxide, alkali metal carbonate, or mixture thereof. The steps of the methods disclosed herein are further illustrated in FIG. 1 and described below. The steps of the methods may contact the dairy membranes from about 1 minute to about 240 minutes, from about 5 minutes to about 180 minutes, from about 15 minutes to about 180 minutes, from about 5 minutes to about 180 minutes, most preferably between about 15 minutes and 180 minutes, In a preferred embodiment, the enzymatic step is for a time between about 1 minute and about 150 minutes, more preferably about 5 minutes to about 140 minutes, still more preferably between about 10 minutes and about 130 minutes; most preferably between about 15 minutes and about 120 minutes. In a preferred embodiment, the surfactant step is for a time between about 1 minute and about 90 minutes, more preferably between about 2 minutes and about 75 minutes, most preferably between about 5 minutes and about 60 minutes.

### Membranes

Disclosed herein are methods of cleaning one or more dairy filtration membranes. The membranes can be microfiltration, ultrafiltration, nanofiltration, and/or reverse osmosis membranes. Microfiltration membranes can include, but are not limited to, Hydranautics SuPro, Synder LX, Synder FR, Alfa Laval GRM 0.1PP, Synder V0.1, Koch Dairy Pro MF-0.1, Alfa Laval FSM0.15, Alfa Laval GRM 0.2PP, Synder V0.2, Alfa Laval FSM0.45. Ultrafiltration membranes can include, but are not limited to, Koch Dairy Pro 5K, Koch HFK-131, Alfa Laval GR61PP, Alfa Laval GR60PP, Synder MK, Alfa Laval GR51PP, Synder MQ, Alfa Laval FS40PP, Synder LY, Synder PY, Synder BY, Koch, HFM-180. Preferably, the filtration membranes are Koch Dairy Pro 5K, Koch HFK-131, Alfa Laval GR61PP, or Alfa Laval GR60PP. The filtration membranes can polymers which include, for example, PES, PS, PVDF, PAN, PA or the like. Preferably, the polymers include PES and PS. The filtration membranes can have an approximate molecular weight cut-off preferably from about 5 kDa to 5000 kDa. The filtration membranes can have an approximate pore size preferably from 0.0005 μm to 0.45 μm.

### Pre-Rinse

Preferably, a membrane is pre-rinsed in a prerinse step 101. The pre-rinse can remove some soils from the membrane, typically loose soils. The rinse is preferably performed with water. The water can be tap water or a water that has been softened. The water can have a hardness of about 20 grains or less, preferably about 15 grains or less, more preferably about 10 grains or less, even more preferably 5 grains or less. Most preferably, the water is distilled water or RO (reverse osmosis) water. The rinse water can be any temperature for which the membrane is compatible. Thus, tap water can be used, room temperature water can used, or heated water can be used so long as it does not exceed the temperature guidelines for the particular membrane. For most membranes, this will be up to 50° C. For high temperature membranes, this can be up to 60° C. or even up to 70° C. For a standard membrane, preferably the temperature of the rinse water is between 20° C. and 50° C., more preferably between 25° C. and 50° C., most preferably between 30° C. and 50° C. for a high temperature membrane, preferably the temperature of the rinse water is between 20° C. and 70° C., more preferably between 25° C. and 70° C., most preferably between 30° C. and 70° C.

### Optional Preclean

Optionally, a membrane can be pre-cleaned with a membrane cleaning detergent in an optional preclean step 102. A membrane cleaning detergent can aid in removing some of the easier to clean soils from the membrane thereby allowing the methods disclosed herein to focus more on the difficult soils. Any suitable membrane cleaning detergent can be employed.

If a preclean step is performed, then a follow-up rinse 103 is performed to remove the membrane cleaning detergent. The rinse is preferably performed with water. The water can be tap water or a water that has been softened. The water can have a hardness of about 20 grains or less, preferably about 15 grains or less, more preferably about 10 grains or less, even more preferably 5 grains or less. Most preferably, the water is distilled water or RO (reverse osmosis) water. The rinse water can be any temperature for which the membrane is compatible. Thus, tap water can be used, room temperature water can used, or heated water can be used so long as it does not exceed the temperature guidelines for the particular membrane. For most membranes, this will be up to 50° C. For high temperature membranes, this can be up to 60° C. or even up to 70° C. For a standard membrane, preferably the temperature of the rinse water is between 20° C. and 50° C., more preferably between 25° C. and 50° C., most preferably between 30° C. and 50° C. for a high temperature membrane, preferably the temperature of the rinse water is between 20° C. and 70° C., more preferably between 25° C. and 70° C., most preferably between 30° C. and 70° C.

### Enzyme Step

The membrane is contacted with an enzyme composition in an enzyme step 104. The enzyme composition comprises a carrier, an enzyme and a buffer. The carrier is preferably water. The water can be tap water, distilled water, or RO (reverse osmosis) water. The water can have a hardness of about 20 grains or less, preferably about 15 grains or less, more preferably about 10 grains or less, even more preferably 5 grains or less. Tap water can be used, room temperature water can used, or heated water can be used so long as it does not exceed the temperature guidelines for the particular membrane. Preferably, the enzyme composition further comprises a chelant. The amount of chelant can be influenced by the hardness of the water.

The pH of the enzyme composition is preferably from about 7.5 to about 11.0, more preferably from about 8.0 to about 10.5, most preferably from about 9 to about 10.

The temperature of the enzyme composition can be any temperature for which the membrane is compatible. For most membranes, this will be up to 50° C. For high temperature membranes, this can be up to 60° C. or even up to 70° C. For a standard membrane, preferably the temperature of the rinse water is between 20° C. and 50° C., more preferably between 25° C. and 50° C., most preferably between 30° C. and 50° C. for a high temperature membrane, preferably the temperature of the rinse water is between 20° C. and 70° C., more preferably between 25° C. and 70° C., most preferably between 30° C. and 70° C.

### Lipase

The enzyme composition comprises a lipase. Any lipase or mixture of lipases, from any source, can be used in the enzyme composition, provided that the selected lipase is stable in a pH range compatible with the type of membrane. For example, the lipase enzymes can be derived from a plant, an animal, or a microorganism such as a fungus or a bacterium. The lipase can be purified or a component of a microbial extract, and either wild type or variant (either chemical or recombinant).

Preferred lipase enzymes include, but are not limited to, the enzymes derived from a Pseudomonas, such as Pseudomonas stutzeri ATCC 19.154, or from a Thermomyces, such as Thermomyces lanuginosus (typically produced recombinantly in Aspergillus oryzae). Most preferably, the lipase comprises a variant of the wild-type Thermomyces lanuginosus lipase and has at least 90% sequence identity to SEQ ID NO 1:

EVSQDLFNQF NLFAQYSAAA YCGKNNDAPA GTNITCTGNA
CPEVEKADAT FLYSFEDSGV GDVTGFLALD NTNKLIVLSF
RGSRSIENWI GNLNFDLKEI NDICSGCRGH DGFTSSWRSV
ADTLRQKVED AVREHPDYRV VFTGHSLGGA LATVAGADLR
GNGYDIDVFS YGAPRVGNRA FAEFLTVQTG GTLYRITHTN
DIVPRLPPRE FGYSHSSPEY WIKSGTLVPV RRRDIVKIEG
IDATGGNNQP NIPDIPAHLW YFGLIGTCL

### Protease

The enzyme composition can further comprise a protease. Any protease or mixture of proteases, from any source, can be used in the enzyme composition, provided that the selected protease is stable in a pH range compatible with the type of membrane. For example, the protease enzymes can be derived from a plant, an animal, or a microorganism such as a yeast, a mold, or a bacterium. Preferred protease enzymes include, but are not limited to, the enzymes derived from Bacillus subtilis, Bacillus licheniformis and Streptomyces griseus. Protease enzymes derived from B. subtilis are most preferred. The protease can be purified or a component of a microbial extract, and either wild type or variant (either chemical or recombinant).

### Buffer

The enzyme compositions comprise a buffer. Preferably the buffer is selected based on the optimal pH for the enzyme(s) in the enzyme composition. Preferred buffers include those suitable for buffering the composition such that it maintains a pH between 7.5 and 11.0. Any suitable buffer achieving this pH can be utilized. In a preferred embodiment, the buffer comprises a carbonate-based buffer, including, but not limited to an alkali metal carbonate, sodium bicarbonate, or a mixture thereof.

The amount of buffer included is the amount needed to retain a pH for optimal enzyme activity. In a preferred embodiment, the buffer is in a concentration of between about

Chelant and/or Sequestrant

The enzyme composition can optionally comprise a chelant and/or sequestrant. As described herein, chelants include compounds that form water soluble complexes with metals. As described herein, sequestrants include compounds that form water insoluble complexes with metals.

Preferred chelants include aminocarboxylates, sodium tripolyphosphate, citrate (in their acid or salt form). Preferred aminocarboxylates include biodegradable aminocarboxylates. Examples of suitable biodegradable aminocarboxylates include: ethanoldiglycine, e.g., an alkali metal salt of ethanoldiglycine, such as disodium ethanoldiglycine (Na₂EDG); methylgylcinediacetic acid (MGDA), e.g., an alkali metal salt of methylgylcinediacetic acid, such as trisodium methylgylcinediacetic acid; ethylenediaminetetraacetic acid (EDTA); iminodisuccinic acid, e.g., an alkali metal salt of iminodisuccinic acid, such as iminodisuccinic acid sodium salt; N,N-bis-(carboxylatomethyl)-L-glutamic acid (GLDA), e.g., an alkali metal salt of N,N-bis(carboxylatomethyl)-L-glutamic acid, such as iminodisuccinic acid sodium salt (GLDA-Na₄); [S—S]-ethylenediaminedisuccinic acid (EDDS), e.g., an alkali metal salt of [S—S]-ethylenediaminedisuccinic acid, such as a sodium salt of [S—S]-ethylenediaminedisuccinic acid; 3-hydroxy-2,2′-iminodisuccinic acid (HIDS), e.g., an alkali metal salt of 3-hydroxy-2,2′-iminodisuccinic acid, such as tetrasodium 3-hydroxy-2,2′-iminodisuccinate.

Some examples of polymeric polycarboxylates suitable for use as sequestering agents include those having a pendant carboxylate (—CO₂) groups and include, for example, polyacrylic acid, maleic/olefin copolymer, acrylic/maleic copolymer, polymethacrylic acid, acrylic acid-methacrylic acid copolymers, hydrolyzed polyacrylamide, hydrolyzed polymethacrylamide, hydrolyzed polyamide-methacrylamide copolymers, hydrolyzed polyacrylonitrile, hydrolyzed polymethacrylonitrile, hydrolyzed acrylonitrile-methacrylonitrile copolymers, and the like.

If included, the chelant and/or sequestrant is preferably in a concentration between about 10 ppm and about 10,000 ppm, more preferably between about 25 ppm and about 5,000 ppm, most preferably between about 50 ppm and about 2,500 pm.

### Surfactant Step

The membrane is contacted with a surfactant composition in a surfactant step 105. The surfactant composition comprises a carrier and a surfactant. The carrier is preferably water. The water can be tap water, distilled water, or RO (reverse osmosis) water. The water can have a hardness of about 20 grains or less, preferably about 15 grains or less, more preferably about 10 grains or less, even more preferably 5 grains or less. Tap water can be used, room temperature water can used, or heated water can be used so long as it does not exceed the temperature guidelines for the particular membrane. In a preferred embodiment, the surfactant composition further comprises additional protease and/or additional buffer. The proteases and buffers described above are appropriate for the surfactant composition. The protease can be the same as that included in the enzyme step or a different protease than used in the enzyme step.

### Surfactant

The surfactant composition comprises a surfactant. Preferred surfactants include, but are not limited to, nonionic surfactants, amphoteric surfactants, a sulfonated surfactant, or a mixture thereof. More preferred, the surfactant comprises an alkyl polyglucoside, an amine oxide, an alcohol ethoxylate, an alkoxylated block copolymer, a sulfonated surfactant, or a mixture thereof. Most preferred, the surfactant comprises an alkyl polyglucoside, an amine oxide, a sulfonated surfactant, or a mixture thereof.

### Amine Oxide

Amine oxides are tertiary amine oxides corresponding to the general formula:

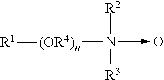

wherein the arrow is a conventional representation of a semi-polar bond; and, R¹, R², and R³ may be aliphatic, aromatic, heterocyclic, alicyclic, or combinations thereof. Generally, for amine oxides of detergent interest, R¹ is an alkyl radical of from about 8 to about 18 carbon atoms; R² and R³ are alkyl or hydroxyalkyl of 1-3 carbon atoms or a mixture thereof; R² and R³ can be attached to each other, e.g. through an oxygen or nitrogen atom, to form a ring structure; R⁴ is an alkaline or a hydroxyalkylene group containing 2 to 3 carbon atoms; and n ranges from 0 to about 20.

Preferred amine oxides can include those selected from the coconut or tallow alkyl di-(lower alkyl) amine oxides, specific examples of which are dodecyldimethylamine oxide, tridecyldimethylamine oxide, tetradecyldimethylamine oxide, pentadecyldimethylamine oxide, hexadecyldimethylamine oxide, heptadecyldimethylamine oxide, octadecyldimethylaine oxide, dodecyldipropylamine oxide, tetradecyldipropylamine oxide, hexadecyldipropylamine oxide, tetradecyldibutylamine oxide, octadecyldibutylamine oxide, bis(2-hydroxyethyl) dodecylamine oxide, bis(2-hydroxyethyl)-3-dodecoxy-1-hydroxypropylamine oxide, dimethyl-(2-hydroxydodecyl)amine oxide, 3,6,9-trioctadecyldimethylamine oxide and 3-dodecoxy-2-hydroxypropyldi-(2-hydroxyethyl)amine oxide.

### Nonionic Surfactants

Preferred nonionic surfactants include, but are not limited to, block copolymers, alcohol alkoxylates, alkoxylated surfactants, reverse EO/PO copolymers, alkylpolyglucosides, alkoxylated amines, fatty acid alkoxylates, fatty amide alkoxylate, alkanoates, and combinations thereof.

Nonionic surfactants are generally characterized by the presence of an organic hydrophobic group and an organic hydrophilic group and are typically produced by the condensation of an organic aliphatic, alkyl aromatic or polyoxyalkylene hydrophobic compound with a hydrophilic alkaline oxide moiety which in common practice is ethylene oxide or a polyhydration product thereof, polyethylene glycol. Practically any hydrophobic compound having a hydroxyl, carboxyl, amino, or amido group with a reactive hydrogen atom can be condensed with ethylene oxide, or its polyhydration adducts, or its mixtures with alkoxylenes such as propylene oxide to form a nonionic surface-active agent. The length of the hydrophilic polyoxyalkylene moiety which is condensed with any particular hydrophobic compound can be readily adjusted to yield a water dispersible or water soluble compound having the desired degree of balance between hydrophilic and hydrophobic properties.

Preferred liquid nonionic surfactants include, but are not limited to:
A. Block polyoxypropylene-polyoxyethylene polymeric compounds based upon propylene glycol, ethylene glycol, glycerol, trimethylolpropane, and ethylenediamine as the initiator reactive hydrogen compound.
B. Condensation products of one mole of a saturated or unsaturated, straight or branched chain alcohol having from about 6 to about 24 carbon atoms with from about 3 to about 50 moles of ethylene oxide. The alcohol moiety can comprise, consist essentially of, or consist of mixtures of alcohols in the above delineated carbon range or it can consist of an alcohol having a specific number of carbon atoms within this range, or it can be a guerbet alcohol ethoxylate.

In addition to ethoxylated carboxylic acids, commonly called polyethylene glycol esters, other alkanoic acid esters formed by reaction with glycerides, glycerin, and polyhydric (saccharide or sorbitan/sorbitol) alcohols have application in this invention for specialized embodiments. All of these ester moieties have one or more reactive hydrogen sites on their molecule which can undergo further acylation or ethylene oxide (alkoxide) addition to control the hydrophilicity of these substances. Care must be exercised when adding these fatty ester or acylated carbohydrates to compositions containing lipase enzymes because of potential incompatibility.
C. The ethoxylated C6-Cis fatty alcohols and C6-Cis mixed ethoxylated and propoxylated fatty alcohols are suitable surfactants for use in the present compositions, particularly those that are water soluble. Suitable ethoxylated fatty alcohols include the C6-C18 ethoxylated fatty alcohols with a degree of ethoxylation of from 3 to 50.
D. Suitable nonionic surfactants suitable for use with the compositions of the present invention include alkoxylated surfactants. Suitable alkoxylated surfactants include EO/PO copolymers, capped EO/PO copolymers, alcohol alkoxylates, capped alcohol alkoxylates, mixtures thereof, or the like. Suitable alkoxylated surfactants for use as solvents include EO/PO block copolymers, such as the Pluronic and reverse Pluronic surfactants; alcohol alkoxylates, capped alcohol alkoxylates, and mixtures thereof.
E. Compounds from (1) which are modified, essentially reversed, by adding ethylene oxide to ethylene glycol to provide a hydrophile of designated molecular weight; and, then adding propylene oxide to obtain hydrophobic blocks on the outside (ends) of the molecule.
F. Suitable nonionic alkylpolysaccharide surfactants, particularly for use in the present compositions include those disclosed in U.S. Pat. No. 4,565,647, Llenado, issued Jan. 21, 1986. These surfactants include a hydrophobic group containing from about 6 to about 30 carbon atoms and a polysaccharide, e.g., a polyglycoside, hydrophilic group containing from about 1.3 to about 10 saccharide units. Any reducing saccharide containing 5 or 6 carbon atoms can be used, e.g., glucose, galactose and galactosyl moieties can be substituted for the glucosyl moieties. (Optionally the hydrophobic group is attached at the 2-, 3-, 4-, etc. positions thus giving a glucose or galactose as opposed to a glucoside or galactoside.) The intersaccharide bonds can be, e.g., between the one position of the additional saccharide units and the 2-, 3-, 4-, and/or 6-positions on the preceding saccharide units.
G. Suitable nonionic surfactants also include the class defined as alkoxylated amines or, most particularly, alcohol alkoxylated/aminated/alkoxylated surfactants. These non-ionic surfactants may be at least in part represented by the general formulae: R²⁰—(PO)_{S}N-(EO)ₜH, R²⁰—(PO)_{S}N-(EO)ₜH(EO)ₜH, and R²⁰—N(EO)ₜH; in which R²⁰ is an alkyl, alkenyl or other aliphatic group, or an alkyl-aryl group of from 8 to 20, preferably 12 to 14 carbon atoms, EO is oxyethylene, PO is oxypropylene, s is 1 to 20, preferably 2-5, t is 1-10, preferably 2-5, and u is 1-10, preferably 2-5. Other variations on the scope of these compounds may be represented by the alternative formula: R²⁰—(PO)_{V}—N [(EO)_{w}H][(EO)_{z}H] in which R²⁰ is as defined above, v is 1 to 20 (e.g., 1, 2, 3, or 4 (preferably 2)), and w and z are independently 1-10, preferably 2-5.
H. Suitable nonionic surfactants also include fatty acid amide alkoxylates. Preferably such surfactants include those having the structural formula R²CON_{R1}Z in which: R1 is H, C₁-C₄ hydrocarbyl, 2-hydroxy ethyl, 2-hydroxy propyl, ethoxy, propoxy group, or a mixture thereof; R² is a C₅-C₃₁ hydrocarbyl, which can be straight-chain; and Z is a polyhydroxyhydrocarbyl having a linear hydrocarbyl chain with at least 3 hydroxyls directly connected to the chain, or an alkoxylated derivative (preferably ethoxylated or propoxylated) thereof. Z can be derived from a reducing sugar in a reductive amination reaction; such as a glycityl moiety.

The alkyl ethoxylate condensation products of aliphatic alcohols with from about 0 to about 25 moles of ethylene oxide are suitable for use in the present compositions. The alkyl chain of the aliphatic alcohol can either be straight or branched, primary or secondary, and generally contains from 6 to 22 carbon atoms.

Fatty acid amide surfactants suitable for use the present compositions include those having the formula: R₆CON(R₇)₂ in which R₆ is an alkyl group containing from 7 to 21 carbon atoms and each R₇ is independently hydrogen, C₁-C₄ alkyl, C₁-C₄ hydroxyalkyl, or —(C₂H₄O)_{X}H, where x is in the range of from 1 to 3.
I. Suitable nonionic surfactants also include nonionic alkanoates. Suitable alkanoates are nonionic esters or salts thereof formed from the reaction of alkanoic acid and an alkanol.
J. Suitable alkylpolyglucosides include quaternized sugar-derived surfactants including a quaternized alkyl polyglucoside or a polyquaternized alkyl polyglucoside, and the like. The quaternary functionalized alkyl polyglucoside is a naturally derived cationic surfactant from alkyl polyglucosides and has a sugar backbone. Quaternary functionalized alkyl polyglucosides have the following representative formula:

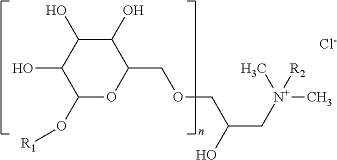

wherein R₁ is an alkyl group having from about 1 to about 22 carbon atoms, and R₂ is CH₃ (CH₂)_{n′} where n′ is an integer ranging from 0-21. Examples of suitable quaternary functionalized alkyl polyglucosides components which can be used in the cleansing compositions according to the present invention include those in which the R₁ alkyl moiety contains primarily about 10-12 carbon atoms, the R₂ group is CH₃ and n is the degree of polymerization of 1-2.

A polyquaternary alkyl polyglucoside is naturally derived from alkyl polyglucosides and has a sugar backbone. Polyquaternary alkyl polyglucosides have the following representative formula:

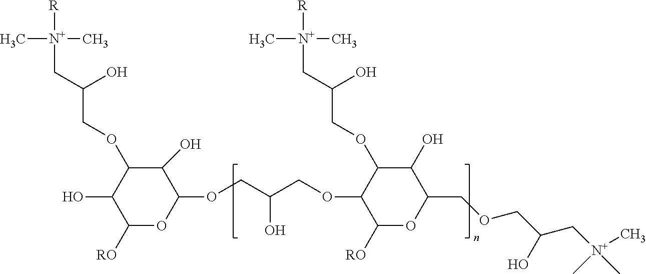

wherein R is an alkyl group having from about 6 to about 22 carbon atoms and n is an integer ranging from 4 to 6. Examples of suitable polyquaternary functionalized alkyl polyglucosides which can be used in the compositions include those in which the R alkyl moiety contains from about 8 to about 12-carbon atoms. In a preferred embodiment the quaternary functionalized alkyl poly glucoside contains primarily about 10-12 carbon atoms.

### Sulfonated Surfactant

The surfactant composition can comprise a sulfonated surfactant. Preferred sulfonated surfactants include sodium capryl sulfonate, sodium lauryl sulfate, linear alkyl benzene sulphonates, sodium dodecyl benzene sulfonate, or a mixture thereof.

The pH of the enzyme composition is preferably from about 7.5 to about 11.0, more preferably from about 8.0 to about 10.5, most preferably from about 8.5 to about 9.5.

The temperature of the enzyme composition can be any temperature for which the membrane is compatible. For most membranes, this will be up to 50° C. For high temperature membranes, this can be up to 60° C. or even up to 70° C. For a standard membrane, preferably the temperature of the rinse water is between 20° C. and 50° C., more preferably between 25° C. and 50° C., most preferably between 30° C. and 50° C. for a high temperature membrane, preferably the temperature of the rinse water is between 20° C. and 70° C., more preferably between 25° C. and 70° C., most preferably between 30° C. and 70° C.

The pH of the surfactant composition is preferably from about 7.5 to about 11.5, more preferably from about 8.0 to about 11.0, most preferably from about 8.5 to about 10.5.

The temperature of the surfactant composition can be any temperature for which the membrane is compatible. Thus, tap water can be used, room temperature water can used, or heated water can be used so long as it does not exceed the temperature guidelines for the particular membrane. For most membranes, this will be up to 50° C. For high temperature membranes, this can be up to 60° C. or even up to 70° C. For a standard membrane, preferably the temperature of the rinse water is between 20° C. and 50° C., more preferably between 25° C. and 50° C., most preferably between 30° C. and 50° C. for a high temperature membrane, preferably the temperature of the rinse water is between 20° C. and 70° C., more preferably between 25° C. and 70° C., most preferably between 30° C. and 70° C.

### Rinse Step

Following the surfactant cleaning step, the membrane is rinsed to remove any excess enzyme, surfactant, buffer, and chelant in a rinse step 106. In particular, it is important to remove the lipase as residual lipase can react to form fatty acid salts which cause bad taste to dairy subsequently processed via the dairy filtration membrane. The rinse is preferably performed with water. The water can be tap water or a water that has been softened. The water can have a hardness of about 20 grains or less, preferably about 15 grains or less, more preferably about 10 grains or less, even more preferably 5 grains or less. Most preferably, the water is distilled water or RO (reverse osmosis) water. The rinse water can be any temperature for which the membrane is compatible. Thus, tap water can be used, room temperature water can used, or heated water can be used so long as it does not exceed the temperature guidelines for the particular membrane. For most membranes, this will be up to 50° C. For high temperature membranes, this can be up to 60° C. or even up to 70° C. For a standard membrane, preferably the temperature of the rinse water is between 20° C. and 50° C., more preferably between 25° C. and 50° C., most preferably between 30° C. and 50° C. for a high temperature membrane, preferably the temperature of the rinse water is between 20° C. and 70° C., more preferably between 25° C. and 70° C., most preferably between 30° C. and 70° C.

### Lipase Acid Deactivation Step

We have found it is critical to deactivate the lipase by unfolding the enzyme protein in a lipase acid deactivation step 107. This provides the technical benefit of the formation of fatty acids salts which cause foul tastes to dairy products. Any residual lipase on the membrane can be deactivated by contacting the membrane with an acidic composition. The acidic composition preferably comprises an acid, an acidic anionic surfactant, or a mixture thereof. The pH of the acid composition is preferably about 2.5 or lower, more preferably about 2.4 or lower, still more preferably about 2.3 or lower, even more preferably about 2.2 or lower, yet more preferably about 2.1 or lower, most preferably about 2.0 or lower or lower than 2.0.

### Acidic Anionic Surfactants

The acidic composition can comprise an acidic anionic surfactant. Anionic surfactants are surface active substances which are categorized by the negative charge on the hydrophile; or surfactants in which the hydrophilic section of the molecule carries no charge unless the pH is elevated to the pKa or above (e.g. carboxylic acids). Some anionic surfactants have an acid pH in solution. Carboxylate, sulfonate, sulfate and phosphate are the polar (hydrophilic) solubilizing groups found in anionic surfactants. Of the cations (counter ions) associated with these polar groups, sodium, lithium and potassium impart water solubility; ammonium and substituted ammonium ions provide both water and oil solubility; and calcium, barium, and magnesium promote oil solubility.

Preferred acidic anionic surfactants, include, but are not limited to, sulfonated surfactants include alkyl sulfonates, the linear and branched primary and secondary alkyl sulfonates, and the aromatic sulfonates with or without substituents. In an aspect, sulfonates include sulfonated carboxylic acid esters. In an aspect, suitable alkyl sulfonate surfactants include C8-C22 alkylbenzene sulfonates, or C10-C22 alkyl sulfonates. In an exemplary aspect, the acidic anionic surfactant comprises an alkyl sulfonate surfactant, most preferably linear alkyl benzene sulfonic acid (LAS). In a further embodiment, the acidic anionic surfactant may alternatively or additionally include diphenylated sulfonates, and/or sulfonated oleic acid. Most preferred acidic anionic surfactants include, but are not limited to, C8-C22 alkylbenzene sulfonates, sulfonated oleic acid, a sulfosuccinate, a secondary alkane sulfonate, or mixtures thereof.

### Acid

The acidic composition can comprise an acid. Preferred acids include organic acids, nitric acid, phosphoric acid, methanesulfonic acid, and mixtures thereof. Preferred organic acids include, but are not limited to, formic acid, acetic acid, glycolic acid, glyoxylic acid, oxalic acid, propionic acid, lactic acid, glyceric acid, malonic acid, tartronic acid, glycidic acid, butanoic acid, 2-methylpropanoic acid, citric acid, and mixtures thereof. Strong acids are not suitable, including, but not limited to, sulfuric acid, hydrochloric acid, and hydrofluoric acid. More preferred acids include nitric acid, phosphoric acid, methanesulfonic acid, lactic acid, citric acid, and mixtures thereof. Most preferred acids include citric acid, lactic acid, and mixtures thereof.

The temperature of the acidic composition can be any temperature for which the membrane is compatible. Thus, tap water can be used, room temperature water can used, or heated water can be used so long as it does not exceed the temperature guidelines for the particular membrane. For most membranes, this will be up to 50° C. For high temperature membranes, this can be up to 60° C. or even up to 70° C. For a standard membrane, preferably the temperature of the rinse water is between 20° C. and 50° C., more preferably between 25° C. and 50° C., most preferably between 30° C. and 50° C. for a high temperature membrane, preferably the temperature of the rinse water is between 20° C. and 70° C., more preferably between 25° C. and 70° C., most preferably between 30° C. and 70° C.

### Rinse Step

Following the lipase deactivation step, the membrane is rinsed to remove any excess acid and/or surfactant in a rinse step 108. The rinse is preferably performed with water. The water can be tap water or a water that has been softened. The water can have a hardness of about 20 grains or less, preferably about 15 grains or less, more preferably about 10 grains or less, even more preferably 5 grains or less. Most preferably, the water is distilled water or RO (reverse osmosis) water. The rinse water can be any temperature for which the membrane is compatible. Thus, tap water can be used, room temperature water can used, or heated water can be used so long as it does not exceed the temperature guidelines for the particular membrane. For most membranes, this will be up to 50° C. For high temperature membranes, this can be up to 60° C. or even up to 70° C. For a standard membrane, preferably the temperature of the rinse water is between 20° C. and 50° C., more preferably between 25° C. and 50° C., most preferably between 30° C. and 50° C. for a high temperature membrane, preferably the temperature of the rinse water is between 20° C. and 70° C., more preferably between 25° C. and 70° C., most preferably between 30° C. and 70° C.

### Optional Alkalinity Step

Optionally, the membrane can be cleaned with an alkalinity step 109. The alkalinity step 109 can aid in further deactivating the lipase by charging portions of the unfolded protein such that it remains unfolded. This step 109 is not required but can aid in deactivation in some contexts. In the alkalinity step 109, the membrane is contacted with an alkaline composition. The alkaline composition preferably comprises an alkali metal hydroxide, alkali metal carbonate, or mixture thereof. Preferred alkali metal hydroxides include sodium hydroxide, potassium hydroxide, or a mixture thereof. Preferred alkali metal carbonates include sodium carbonate, potassium carbonate, or a mixture thereof. In addition to the alkali metal hydroxide and/or alkali metal carbonate, the alkaline composition can optionally further comprise an alkali metal silicate, a metasilicate, sesquicarbonate, organic sources of alkalinity or mixtures thereof.

Organic alkalinity sources are often strong nitrogen bases including, for example, ammonia (ammonium hydroxide), amines, alkanolamines, and amino alcohols. Typical examples of amines include primary, secondary or tertiary amines and diamines carrying at least one nitrogen linked hydrocarbon group, which represents a saturated or unsaturated linear or branched alkyl group having at least 10 carbon atoms and preferably 16-24 carbon atoms, or an aryl, aralkyl, or alkaryl group containing up to 24 carbon atoms, and wherein the optional other nitrogen linked groups are formed by optionally substituted alkyl groups, aryl group or aralkyl groups or polyalkoxy groups. Typical examples of alkanolamines include monoethanolamine, monopropanolamine, diethanolamine, dipropanolamine, triethanolamine, tripropanolamine and the like. Typical examples of amino alcohols include 2-amino-2-methyl-1-propanol, 2-amino-1-butanol, 2-amino-2-methyl-1,3-propanediol, 2-amino-2-ethyl-1,3-propanediol, hydroxymethyl aminomethane, and the like.

The pH of the alkaline composition used in the optional alkalinity step is preferably from about 9.5 to about 12.0, more preferably from about 9.7 to about 11.8, most preferably from about 10.0 to about 11.5.

If an alkalinity step is performed, then a follow-up rinse step 110 is performed to remove the alkaline composition. The rinse is preferably performed with water. The water can be tap water or a water that has been softened. The water can have a hardness of about 20 grains or less, preferably about 15 grains or less, more preferably about 10 grains or less, even more preferably 5 grains or less. Most preferably, the water is distilled water or RO (reverse osmosis) water. The rinse water can be any temperature for which the membrane is compatible. Thus, tap water can be used, room temperature water can used, or heated water can be used so long as it does not exceed the temperature guidelines for the particular membrane. For most membranes, this will be up to 50° C. For high temperature membranes, this can be up to 60° C. or even up to 70° C. For a standard membrane, preferably the temperature of the rinse water is between 20° C. and 50° C., more preferably between 25° C. and 50° C., most preferably between 30° C. and 50° C. for a high temperature membrane, preferably the temperature of the rinse water is between 20° C. and 70° C., more preferably between 25° C. and 70° C., most preferably between 30° C. and 70° C.

### Preferred Embodiments

The present disclosure is further defined by the following numbered embodiments:
1. A method of cleaning a dairy filtration membrane comprising:
(a) contacting the dairy filtration membrane with an enzyme composition, wherein the enzyme composition comprises a lipase and a buffer, wherein the pH of the enzyme composition is from about 7.5 to about 11.0;
(b) contacting the dairy filtration membrane with a surfactant composition, wherein the surfactant composition comprises an alkyl polyglucoside, an alkyl polypentoside, an amine oxide, an alcohol ethoxylate, an alkoxylated block copolymer, a sulfonated surfactant, or a mixture thereof; wherein the pH of the surfactant composition is from about 7.5 to about 11.0;
(c) rinsing the dairy filtration membrane;
(d) contacting the dairy filtration membrane with an acidic composition, wherein the acidic composition comprises an acid, an acidic anionic surfactant, or a mixture thereof; wherein the pH of the acidic composition is about 2.5 or lower; wherein the acidic composition deactivates the lipase; and
(e) rinsing the dairy filtration membrane.
2. The method of paragraph 1, wherein the lipase comprises a lipase derived from a Pseudomonas or a Humicola.
3. The method of any one of paragraphs 1-2, wherein the enzyme composition further comprises a protease.
4. The method of any one of paragraphs 1-3, wherein the surfactant composition further comprises a protease.
5. The method of paragraph 3 or 4, wherein the protease comprises a protease derived from Bacillus subtilis, Bacillus licheniformis and Streptomyces griseus.
6. The method of any one of paragraphs 1-5, wherein the buffer comprises an alkali metal hydroxide, alkali metal carbonate, sodium bicarbonate, or a mixture thereof.
7. The method of any one of paragraphs 1-6, wherein the enzyme composition comprises a chelant.
8. The method of paragraph 7, wherein the chelant comprises an aminocarboxylic acid or salt thereof, sodium tripolyphosphate, citric acid or a salt thereof, or a mixture thereof.
9. The method of any one of paragraphs 1-8, wherein the acidic composition comprises a linear alkyl sulfonate, linear alkyl sulfate, a branched alkyl sulfonate, a branched alkyl sulfate, a substituted aromatic sulfonate, an unsubstituted aromatic sulfonate, an organic acid, nitric acid, phosphoric acid, methane sulfonic acid, or mixture thereof.
10. The method of any one of paragraphs 1-9, wherein the surfactant composition comprises one or more of a C₅, C₁₂-C₁₄ alkylpolypentoside, a C₈-C₁₆ alkylpolyglycoside, an EO PO block copolymer, nonylphenol ethoxylate, lauryl dimethylamine oxide, linear alkylbenzene sulfonate, 2-ethyl-hexyl alkoxylate, a C₆ alkyl polyglycoside, a secondary n-alkyl sulfonate, sodium lauryl sulfonate; sodium C₁₂-14 alcohol sulfate, or a mixture thereof.
11. The method of any one of paragraphs 1-10, further comprising
(f) contacting the dairy filtration membrane with an alkaline composition; wherein the alkaline composition has a pH from about 9.5 to about 12.0; and
(g) rinsing the dairy filtration membrane.
12. The method of paragraph 11, wherein the alkaline composition comprises an alkali metal hydroxide, alkali metal carbonate, an alkali metal silicate, an organic alkalinity source, or mixture thereof.
13. The method of any one of paragraphs 1-12, further comprising the step of pre-rinsing the dairy filtration membrane before step (a).
14. The method of any one of paragraphs 1-13, further comprising, before step (a), the step of contacting the dairy filtration membrane with a membrane detergent composition and subsequently rinsing the dairy filtration membrane.
15. The method of any one of paragraphs 1-14, wherein the dairy filtration membrane comprises a microfiltration membrane, an ultrafiltration membrane, nanofiltration membrane, and/or a reverse osmosis membrane.
16. A method of cleaning a dairy filtration membrane comprising:
(a) contacting the dairy filtration membrane with an enzyme composition, wherein the enzyme composition comprises a lipase, a protease, and a buffer, wherein the pH of the enzyme composition is from about 8.0 to about 10.5; wherein the dairy filtration membrane comprises a microfiltration membrane, an ultrafiltration membrane, nanofiltration membrane, and/or a reverse osmosis membrane;
(b) contacting the dairy filtration membrane with a surfactant composition, wherein the surfactant composition comprises an alkyl polyglucoside, an alkyl polypentoside, an amine oxide, an alcohol ethoxylate, an alkoxylated block copolymer, a sulfonated surfactant, or a mixture thereof; wherein the pH of the surfactant composition is from about 8.0 to about 10.5;
(c) rinsing the dairy filtration membrane;
(d) contacting the dairy filtration membrane with an acidic composition, wherein the acidic composition comprises a linear alkyl sulfonate, a branched alkyl sulfonate, a substituted aromatic sulfonate, an unsubstituted aromatic sulfonate, an organic acid, nitric acid, phosphoric acid, methanesulfonic acid, or mixture thereof, or a mixture thereof; wherein the pH of the acidic composition is about 2.5 or lower; wherein the acidic composition deactivates the lipase; and
(e) rinsing the dairy filtration membrane.
17. The method of paragraph 16, further comprising
(f) contacting the dairy filtration membrane with an alkaline composition; wherein the alkaline composition comprises an alkali metal hydroxide, alkali metal carbonate, an alkali metal silicate, an organic alkalinity source, or mixture thereof; wherein the alkaline composition has a pH from about 10.0 to about 12.0; and
(g) rinsing the dairy filtration membrane.
18. The method of paragraph 17, wherein the alkaline composition comprises an alkali metal hydroxide, alkali metal carbonate, or mixture thereof.
19. The method of any one of paragraphs 16-18, wherein the lipase comprises a lipase derived from a Pseudomonas or a Humicola; wherein the protease comprises a protease derived from Bacillus subtilis, Bacillus licheniformis and Streptomyces griseus; and wherein the buffer comprises an alkali metal carbonate, sodium bicarbonate, or a mixture thereof.
20. The method of any one of paragraphs 16-19, wherein the enzyme composition further comprises a chelant; wherein the chelant comprises an aminocarboxylic acid or salt thereof, sodium tripolyphosphate, citric acid or a salt thereof, or a mixture thereof.
21. The method of any one of paragraphs 16-20, wherein the surfactant composition further comprises a protease; wherein the protease comprises a protease derived from Bacillus subtilis, Bacillus licheniformis and Streptomyces griseus.
22. The method of any one of paragraphs 16-21, wherein the surfactant composition comprises one or more of a C₅, C₁₂-C₁₄ alkylpolypentoside, a C₈-C₁₆ alkylpolyglycoside, an EO PO block copolymer, nonylphenol ethoxylate, lauryl dimethylamine oxide, linear alkylbenzene sulfonate, 2-ethyl-hexyl alkoxylate, a C₆ alkyl polyglycoside, a secondary n-alkyl sulfonate, sodium lauryl sulfonate; sodium C₁₂₋₁₄ alcohol sulfate, or a mixture thereof.
23. The method of any one of paragraphs 1-22, wherein the lipase comprises a variant of the wild-type Thermomyces lanuginosus lipase
24. The method of any one of paragraphs 1-23, wherein the lipase has at least 90% sequence identity to SEQ ID NO 1.

### EXAMPLES

Embodiments of the present disclosure are further defined in the following non-limiting Examples. It should be understood that these Examples, while indicating certain embodiments of the disclosure, are given by way of illustration only. From the above discussion and these Examples, one skilled in the art can ascertain the essential characteristics of this disclosure, and without departing from the spirit and scope thereof, can make various changes and modifications of the embodiments of the disclosure to adapt it to various usages and conditions. Thus, various modifications of the embodiments of the disclosure, in addition to those shown and described herein, will be apparent to those skilled in the art from the foregoing description. Such modifications are also intended to fall within the scope of the appended claims. Multiple commercially available lipase compositions were employed. The specific lipase information is not known as they are proprietary formulas from Novozymes. The three are identified herein as Lipase A, Lipase B, and Lipase C. Each of Lipase A, Lipase B, and Lipase C is a variant of the wild-type Thermomyces lanuginosus lipase and have at least a 90% sequence identity to SEQ ID NO: 1.

### Example 1

Fat is a critical challenge in dairy production. Studies of cleaned and uncleaned field membranes showed a factor of 10 times higher fouling load of fats, especially high-melting triglycerides compared to protein foulants present on the membranes. Fat and protein soils are difficult to clean during a cleaned-in-place regime, and due to the higher melting points of the fat far beyond the cleaned-in place temperature, high melting fat accumulates over the membrane's lifetime. Incomplete removal of membrane fouling impacts membrane production performance and is a significant factor in overall membrane health. Triglyceride fats are challenging to remove and build up on the membrane over time. Triglyceride fats can be degraded via a catalytic reaction with lipase. The lipase molecule unfolds the three-dimensional structure of the triglyceride fats, which creates a loss of active sites, as can be seen by the diagram below.

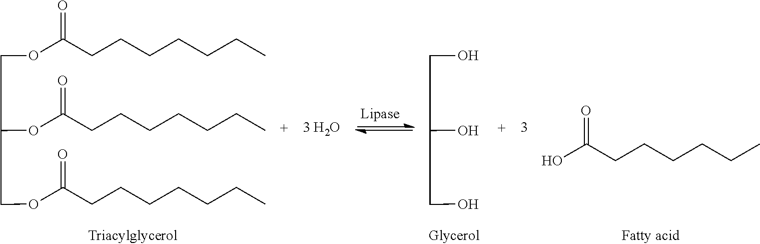

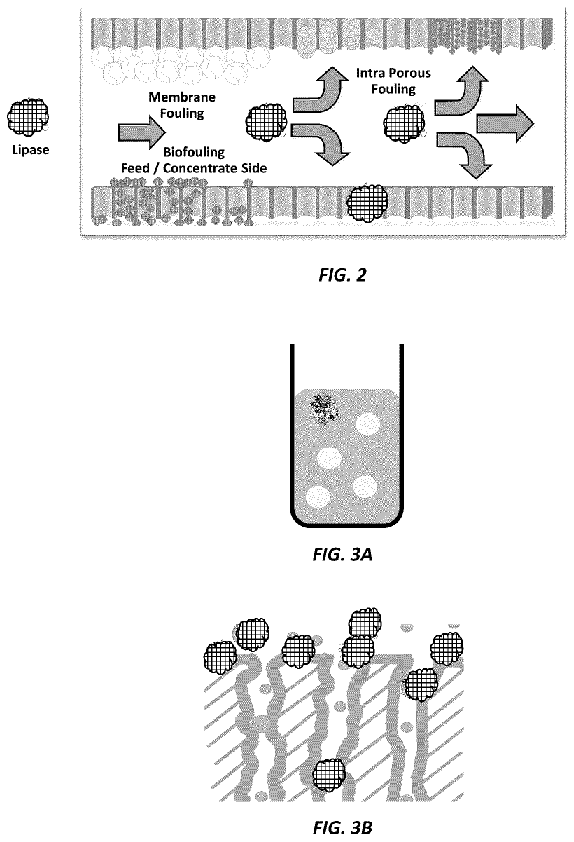

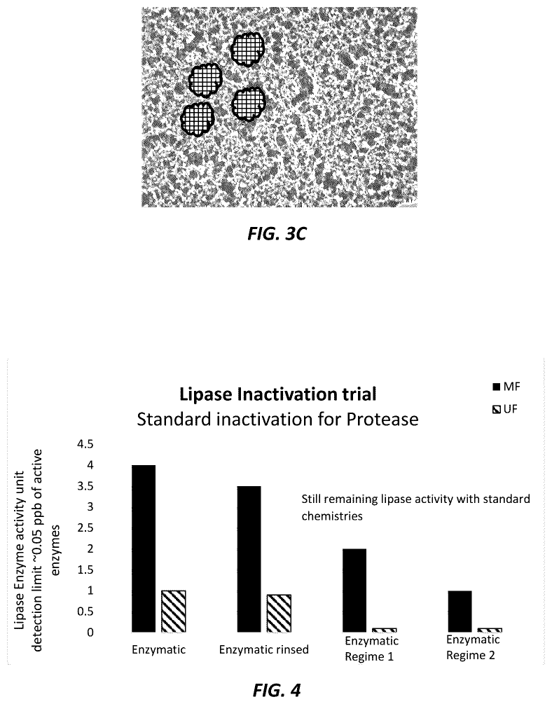

FIG. 2 demonstrates that lipases, although able to remove the fats from the membranes, also stick to the membrane surface, as well as in the pores. FIG. 3C shows an SEM surface picture of a microfiltration membrane. Inactivation of the lipases is more difficult in a membrane filtration system (FIG. 3B) compared to a liquid solution (FIG. 3A) scenario. In the upper membrane surface, lipases interact with the membrane pores. Lipolytic enzymes are designed to be stable in high alkaline formulations for e.g., laundry applications. These formulations contain chelates, oxidizers (NaOCI e.g.) and therefore is very difficult to inactivate. Current cleaning regimes do not completely remove all the foluing components from all membrane types and do not address the full fouling phenomena. Lipases are surface active and stick to the membrane surface and cannot be rinsed away with water. Lipases cannot be inactivated with acids or alkaline. Oxidizers likewise cannot fully inactivate lipase (stability against oxidation).

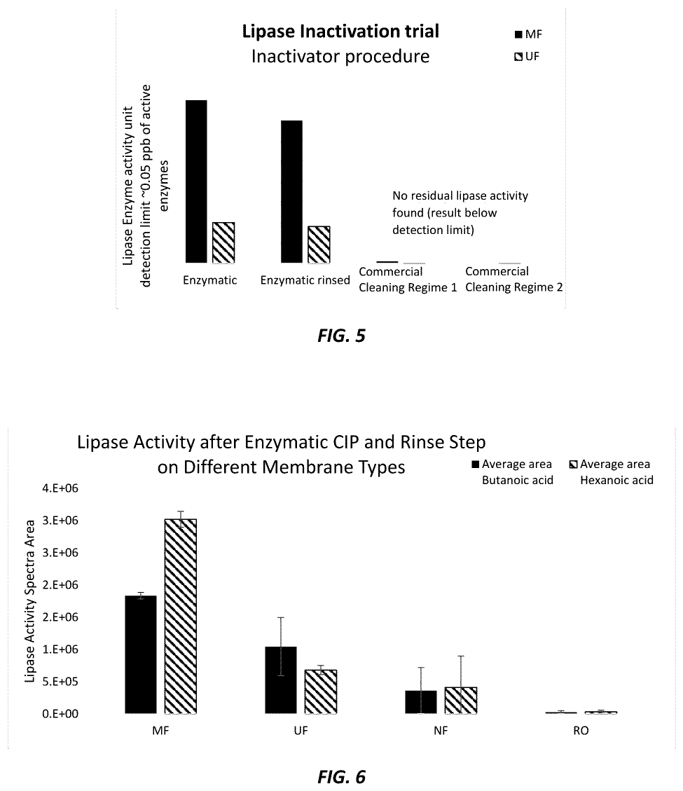

Exemplary cleaning regimes were tested against current regular enzymatic cleaning regimes for their effectiveness in inactivating lipase in microfiltration and ultrafiltration membranes. FIGS. 4 and 5 demonstrate cleaning regime efficacy. FIG. 4 compares 5 standard inactivation procedures (Enzymatic, Enzymatic rinsed, enzymatic regime 1, and enzymatic regime 2). As can be seen, the enzymatic cleaning regimes still show significant amounts of lipase enzyme activity in both the microfiltration and ultrafiltration membranes when compared to the Commercial enzymatic cleaning regimes. Yet, the stated cleaning regimes still have lipase remaining after cleaning. FIG. 5 compares the enzymatic cleaning regimes with the exemplary cleaning regimes (commercial cleaning regimes 1 and 2). As shown by the graph of FIG. 5, the exemplary cleaning regimes show little to no remaining lipase enzymatic activity. Only in the microfiltration membrane utilizing the exemplary cleaning regime without the optional alkaline cleaning step was there any detection. In this test, it is demonstrated that anionic surfactants in the exemplary cleaning regime can unfold lipase in acidic environments. The combination of an additional alkaline step helps to reduce surfactant residues and has a positive contribution to the inactivation procedure.

As shown by the figures, the type of membrane impacts the activity of the lipase on the membrane. The lipase molecule is approximately 30 kDa. Therefore, based purely on size, it will pass or permeate most microfiltration membranes, whereas it would be rejected by ultrafiltration, nanofiltration, and reverse osmosis membranes. If lipase can enter the structure of a microfiltration membrane, the larger internal pore area interacts with the lipase causing more lipase molecules sticking to the membrane, which also makes a rinse out more difficult. For ultrafiltration membranes, lipase could interact with the pores on the membrane surface. As the membrane surface becomes smoother (i.e., smaller pore size) as more the lipase interaction with the membranes decreases. FIG. 6 illustrates these differences between the microfiltration, ultrafiltration, nanofiltration, and reverse osmosis membranes.

### Example 2

Exemplary surfactants to be utilized in an exemplary cleaning regime were tested for cleaning performance of butterfat over time. Polysulfone coupons were cleaned with exemplary surfactants for 10 minutes and 20 minutes at 50° C. Table 3 compares the exemplary surfactants at 9.5 and 11 pH for cleaning performance at 10 minutes at 50° C. Table 4 additionally compares the exemplary surfactants at 9.5 and 11 pH for cleaning performance at 20 minutes. As can be seen in Tables 3, nonylphenol ethoxylate (NPE) at 600 ppm at either 9.5 or 11 pH is the best performing surfactant for removing butterfat at 10 minutes. Additionally, as can be seen in Table 4, NPE at 600 ppm at 11 pH for 20 minutes removed approximately 100% of the soil.

TABLE 3

| | | | | | Add'l | |
|---|---|---|---|---|---|---|
| | | | Surfactant | | Ingreds. | |
| | Time | | Conc. | Add'l | Conc. | % Soil |
| pH | [min] | Chemical Name | [ppm] | Ingreds. | [ppm] | Removal |
| 9.5 | 10 | | 0 | Lipase A | 800 | 23 |
| 11 | 10 | | 0 | NaOH | dosed | 28 |
| | | | | | pH | |
| 9.5 | 10 | | 0 | Carbonate | 5000 | 29 |
| | | | | Buffer | | |
| 11 | 10 | | 0 | NaOH | dosed to | 30 |
| | | | | | pH | |
| 11 | 10 | Linear alkylbenzene | 600 | | | 39 |
| | | sulfonate | | | | |
| 11 | 10 | lauryl | 600 | | | 48 |
| | | dimethylamine | | | | |
| | | oxide | | | | |
| 11 | 10 | lauryl | 600 | | | 48 |
| | | dimethylamine | | | | |
| | | oxide | | | | |
| 9.5 | 10 | lauryl | 600 | | | 56 |
| | | dimethylamine | | | | |
| | | oxide | | | | |
| 11 | 10 | Alkylpolypentoside | 600 | | | 61 |
| | | C₁₂-C₁₄ | | | | |
| 9.5 | 10 | Alkylpolypentoside | 600 | | | 71 |
| | | C₁₂-C₁₄ | | | | |
| 11 | 10 | Alkylpolypentoside | 600 | | | 73 |
| | | C₁₂-C₁₄ | | | | |
| 9.5 | 10 | 2-ethyl-hexyl | 1000 | | | 74 |
| | | alkoxylate | | | | |
| 9.5 | 10 | Alkylpolypentoside | 600 | | | 76 |
| | | C₁₂-C₁₄ | | | | |
| 11 | 10 | 2-ethyl-hexyl | 1000 | | | 78 |
| | | alkoxylate | | | | |
| 9.5 | 10 | Alkylpolypentoside | 420 | | | 80 |
| | | C₁₀-C₁₂ | | | | |
| 11 | 10 | Alkylpolypentoside | 600 | | | 80 |
| | | C₁₂-C₁₄ | | | | |
| 9.5 | 10 | Blend of C₁₀-C₁₂ | 420 | | | 80 |
| | | alkylpolypentoside | | | | |
| | | and C₈-C₁₀ | | | | |
| | | alkylpolyglycoside | | | | |
| 9.5 | 10 | Alkylpolypentoside | 420 | | | 81 |
| | | C₁₀-C₁₂ | | | | |
| 9.5 | 10 | Alkylpolypentoside | 420 | | | 82 |
| | | C5, C12-C14 | | | | |
| 9.5 | 10 | Alkylpolypentoside | 420 | | | 83 |
| | | C₅, C₁₂-C₁₄ | | | | |
| 9.5 | 10 | Alkylpolypentoside | 450 | Carbonate | 1000 | 83 |
| | | C5, C12-C14 | | Buffer | | |
| 9.5 | 10 | Alkylpolypentoside | 600 | | | 88 |
| | | C₅, C₁₂-C₁₄ | | | | |
| 11 | 10 | Alkylpolypentoside | 600 | | | 92 |
| | | C₅, C₁₂-C₁₄ | | | | |
| 11 | 10 | nonylphenol | 600 | | | 93 |
| | | ethoxylate | | | | |
| 9.5 | 10 | Alkylpolypentoside | 600 | | | 93 |
| | | C₅, C₁₂-C₁₄ | | | | |
| 9.5 | 10 | nonylphenol | 600 | | | 95 |
| | | ethoxylate | | | | |
| 11 | 10 | nonylphenol | 600 | | | 96 |
| | | ethoxylate | | | | |

TABLE 4

| | | | | | Add'1 | | |
|---|---|---|---|---|---|---|---|
| | | | Surfactant | | Ingreds. | | |
| | Time | | use Conc | Add'1 | Conc. | | % Soil |
| pH | [min] | Chemical Name | [ppm] | Ingreds. | [ppm] | Procedure | Removal |
| 9.5 | 20 | | | Carbonate | 500 | One Pot | 8 |
| | | | | buffer | | | |
| 11 | 20 | linear alkylbenzene | 600 | | | One Pot | 15 |
| | | sulfonate | | | | | |
| 11 | 20 | dimethyl lauryl amine | 600 | | | One Pot | 54 |
| | | oxide | | | | | |
| 11 | 20 | Alkylpolypentoside | 600 | | | One Pot | 64 |
| | | C₁₂-C₁₄ | | | | | |
| 11 | 20 | 2-ethyl-hexyl alkoxylate | 600 | | | One Pot | 65 |
| 9.5 | 20 | | | Lipase A | 600 | One Pot | 69 |
| 9.5 | 20 | Alkylpolypentoside C₅, | 600 | | | One Pot | 71 |
| | | C₁₂-C₁₄ | | | | | |
| 9.5 | 20 | Alkylpolypentoside C₅, | 450 | | | One Pot | 75 |
| | | C₁₂-C₁₄ | | | | | |
| 11 | 20 | 2-ethyl-hexyl alkoxylate | 1000 | | | One Pot | 85 |
| 9.5 | 20 | Alkylpolypentoside C₅, | 600 | Lipase A | 450 | Top Up | 89 |
| | | C₁₂-C₁₄ | | | | | |
| 9.5 | 20 | Alkylpolypentoside C₅, | 600 | Lipase A | 600 | Top Up | 90 |
| | | C₁₂-C₁₄ | | | | | |
| 9.5 | 20 | 2-ethyl-hexyl alkoxylate | 1000 | Lipase A | 450 | Top Up | 90 |
| 9.5 | 20 | Alkylpolypentoside C₅, | 450 | Lipase A | 450 | Top Up | 90 |
| | | C₁₂-C₁₄ | | | | | |
| 9.5 | 20 | Alkylpolypentoside | 600 | Lipase A | 450 | Top Up | 91 |
| | | C₁₂-C₁₄ | | Carbonate | 1000 | | |
| | | | | Buffer | | | |
| 9.5 | 20 | Alkylpolypentoside C₅, | 600 | Lipase A | 200 | Top Up | 93 |
| | | C₁₂-C₁₄ | | | | | |
| 9.5 | 20 | 2-ethyl-hexyl alkoxylate | 1000 | Lipase A | 200 | Top Up | 94 |
| 9.5 | 20 | Alkylpolypentoside | 600 | Lipase A | 450 | Top Up | 96 |
| | | C₁₂-C₁₄ | | Carbonate | 1000 | | |
| | | | | Buffer | | | |
| 9.5 | 20 | Alkylpolypentoside | 600 | Lipase A | 450 | Top Up | 98 |
| | | C₁₂-C₁₄ | | Carbonate | 1000 | | |
| | | | | Buffer | | | |
| 11 | 20 | nonylphenol ethoxylate | 600 | | | One Pot | 99 |
| 11 | 20 | nonylphenol ethoxylate | 600 | | | | 101 |

### Example 3

Exemplary surfactants, applied all at once (OnePot) or in a piece-wise manner (TopUp), were tested for cleaning performance of ghee. Polysulfone coupons, having the dimensions 1″×3″, and 0.06″ thick, were conditioned by soaking in methyl alcohol for 30 seconds, then placed in 60° C. oven for 30 minutes before weighing on an analytical balance. Ghee is soiled on coupons at 0.0250-0.0300 g in a “c” pattern (seven “c” were made on each coupon with a 1″ foam paint brush). Coupons were then allowed to dry overnight.

Exemplary surfactants applied in the TopUp procedure start with DI water being heated to 50° C. by mixing with a 1.5″ stir bar at 240 RPM in a 1000 mL beaker. When the temperature is reached, 7500 ppm of a buffer solution is added and allowed to mix for approximately 1 minute to achieve a pH of 9.5. 600 mL of a surfactant solution and 150 ppm of a lipase is added to the solution. Coupons are then added to the pot with metal hanger. After 10 minutes, the exemplary product is added to the solution using approximately 1 g of DI water. After an additional 10 minutes the metal hanger with the coupons is removed and coupons are dipped in room temperature DI water once and allowed to dry overnight.

Exemplary surfactants applied in the OnePot procedure start with DI water being heated to 50° C. by mixing with a 1.5″ stir bar at 240 RPM in a 1000 ml beaker. When the temperature is reached, 7500 ppm of a buffer solution is added and allowed to mix for approximately 1 minute to achieve a pH of 9.5. Total volume of the surfactant solution is 600 mL and 150 ppm of lipase is added. Coupons are then added to the pot with metal hanger. After 20 minutes, the metal hanger with the coupons is removed and coupons are dipped in room temperature DI water once and allowed to dry overnight.

Table 5 shows % ghee removal results (% SR) of the exemplary surfactants utilizing both procedures. Without the presence of enzyme, the procedures are equivalent, or One Pot procedure performs better. This is not surprising as the One Pot procedure has surfactant for 20 minutes vs. 10 minutes with Top Up.

TABLE 5

| Procedure | Surfactants | % SR |
|---|---|---|
| TopUp | C₁₀-C₁₂ alkylpolypentoside, 600 ppm | 34.9 |
| TopUp | blend of C₁₀-C₁₂ alkylpolypentoside | 50.1 |
| | and C₆ alkyl polyglycoside, 150 ppm | |
| TopUp | alkylpolyglycoside, 600 ppm | 33.9 |
| TopUp | nonylphenol ethoxylate, 600 ppm | 73.8 |
| TopUp | lauryl dimethylamine oxide, 600 ppm | 30.8 |
| TopUp | linear alkylbenzene sulfonate, 600 ppm | 29.6 |
| TopUp | 2-ethyl-hexyl alkoxylate, 600 ppm | 47.4 |
| TopUp | 2-ethyl-hexyl alkoxylate, 1000 ppm | 64.2 |
| TopUp | secondary n-alkyl sulfonate, 600 ppm | 8.3 |
| TopUp | blend of sodium lauryl sulfonate and | 37.0 |
| | sodium C₁₂₋₁₄ alcohol sulfate, 600 ppm | |
| TopUp | EO PO block copolymer, 600 ppm | 75.6 |
| TopUp | C₆ alkyl polyglycoside, 600 ppm | 16.2 |
| OnePot | C₁₀-C₁₂ alkylpolypentoside, 600 ppm | 44.7 |
| OnePot | C₈-C₁₆ alkylpolyglycoside, 600 ppm | 51.5 |
| OnePot | NPE 9.5, 600 ppm | 73.0 |
| OnePot | lauryl dimethylamine oxide, 600 ppm | 36.6 |
| OnePot | linear alkylbenzene sulfonate, 600 ppm | 26.9 |
| OnePot | 2-ethyl-hexyl alkoxylate, 600 ppm | 64.6 |
| OnePot | 2-ethyl-hexyl alkoxylate, 1000 ppm | 74.8 |
| OnePot | secondary n-alkyl sulfonate, 600 ppm | 11.7 |
| OnePot | blend of sodium lauryl sulfonate and | 33.8 |
| | sodium C₁₂₋₁₄ alcohol sulfate, 600 ppm | |
| OnePot | EO PO block copolymer, 600 ppm | 73.8 |
| OnePot | C₆ alkyl polyglycoside, 600 ppm | 12.9 |

Table 6 shows % ghee removal results (% SR) of the same exemplary surfactants utilizing both procedures, which include enzymes. Enzymes were added initially with the DI water in the 1000 mL beaker, heated to 50° C., and stirred with the 1.5″ stir bar at 240 RPM. The procedures are the same as above. When the enzyme is included in the exemplary procedures, there is no synergy for alkoxylates or short chain alkyl polyglycoside surfactants, which effects the removal results.

TABLE 6

| Procedure | Surfactants | % SR |
|---|---|---|
| TopUp | C₁₀-C₁₂ alkylpolypentoside, 600 ppm | 64.55 |
| TopUp | C₈-C₁₆ alkylpolyglycoside, 600 ppm | 65.18 |
| TopUp | nonylphenol ethoxylate, 600 ppm | 75.12 |
| TopUp | lauryl dimethylamine oxide, 600 ppm | 54.75 |
| TopUp | linear alkylbenzene sulfonate, 600 ppm | 56.11 |
| TopUp | 2-ethyl-hexyl alkoxylate, 600 ppm | 57.28 |
| TopUp | 2-ethyl-hexyl alkoxylate, 1000 ppm | 56.05 |
| TopUp | secondary n-alkyl sulfonate, 600 ppm | 27.02 |
| TopUp | blend of sodium lauryl sulfonate and | 53.47 |
| | sodium C₁₂₋₁₄ alcohol sulfate, 600 ppm | |
| TopUp | EO PO block copolymer, 600 ppm | 75.07 |
| TopUp | C₆ alkyl polyglycoside, 600 ppm | 20.40 |
| OnePot | C₁₀-C₁₂ alkylpolypentoside, 600 ppm | 44.23 |
| OnePot | blend of C₁₀-C₁₂ alkylpolypentoside, 600 | 46.45 |
| | ppm and C₆ alkyl polyglycoside, 150 ppm | |
| OnePot | C₈-C₁₆ alkylpolyglycoside, 600 ppm | 48.10 |
| OnePot | nonylphenol ethoxylate, 600 ppm | 78.21 |
| OnePot | lauryl dimethylamine oxide, 600 ppm | 30.10 |
| OnePot | linear alkylbenzene sulfonate, 600 ppm | 33.04 |
| OnePot | 2-ethyl-hexyl alkoxylate, 600 ppm | 62.85 |
| OnePot | 2-ethyl-hexyl alkoxylate, 1000 ppm | 66.66 |
| OnePot | Secondary n-alkyl sulfonate, 600 ppm | 13.48 |
| OnePot | blend of sodium lauryl sulfonate and | 29.67 |
| | sodium C₅, C₁₂₋₁₄ alcohol sulfate, 600 ppm | |
| OnePot | EO PO block copolymer, 600 ppm | 78.87 |
| OnePot | C₆ alkyl polyglycoside, 600 ppm | 22.58 |

### Example 4

The exemplary procedures of Example 3 are tested below. Tables 7-11 test how the % soil removal (% SR) is affected by enzyme concentration (Table 7), temperature (Table 8), water type (Table 9), enzyme type (Table 10), and surfactant concentration (Table 11).

Table 7 uses one enzyme (Lipase A) at 4 concentrations (0, 25, 50, and 150 ppm) utilizing the TopUp and OnePot procedures as described in Example 3. Table 7 shows that the TopUp procedure utilizing any concentration of the enzyme is better at removing ghee than the OnePot procedure using or not using an enzyme.

TABLE 7

| | | Surf | Enzyme | |
|---|---|---|---|---|
| | | Conc | Conc | |
| Procedure | Surfactant | (ppm) | (ppm) | % SR |
| TopUp | C₁₀-C₁₂ alkylpolypentoside | 600 | 0 | 30.6 |
| TopUp | C₁₀-C₁₂ alkylpolypentoside | 600 | 25 | 62.6 |
| TopUp | C₁₀-C₁₂ alkylpolypentoside | 600 | 50 | 59.5 |
| TopUp | C₁₀-C₁₂ alkylpolypentoside | 600 | 150 | 67.5 |
| OnePot | C₁₀-C₁₂ alkylpolypentoside | 600 | 0 | 34.9 |
| OnePot | C₁₀-C₁₂ alkylpolypentoside | 600 | 25 | 32.8 |
| OnePot | C₁₀-C₁₂ alkylpolypentoside | 600 | 50 | 40.2 |
| OnePot | C₁₀-C₁₂ alkylpolypentoside | 600 | 150 | 34.5 |

Table 8 uses the same surfactant, surfactant concentration, enzyme, and enzyme concentration, utilizing the TopUp and OnePot procedures as described in Example 2 at different temperatures (30, 40, and 50° C.). The enzyme used in Table 8 is Lipase A. Table 8 shows the temperature equally affects the performance of the surfactant/enzyme in either the TopUp or OnePot procedures, with the best performance being the TopUp procedure at 50° C.

TABLE 8

| | | Surf | Enzyme | | |
|---|---|---|---|---|---|
| Proce- | | Conc | Conc | | |
| dure | Surfactant | (ppm) | (ppm) | % SR | Temp |
| TopUp | C₁₀-C₁₂ alkylpolypentoside | 600 | 150 | 35.8 | 30° C. |
| TopUp | C₁₀-C₁₂ alkylpolypentoside | 600 | 150 | 45.7 | 40° C. |
| TopUp | C₁₀-C₁₂ alkylpolypentoside | 600 | 150 | 67.5 | 50° C. |
| OnePot | C₁₀-C₁₂ alkylpolypentoside | 600 | 150 | 34.4 | 30° C. |
| OnePot | C₁₀-C₁₂ alkylpolypentoside | 600 | 150 | 40.6 | 40° C. |
| OnePot | C₁₀-C₁₂ alkylpolypentoside | 600 | 150 | 34.5 | 50° C. |

Table 9 uses the same surfactant, surfactant concentration, enzyme, but different enzyme concentrations, and utilizing the TopUp and OnePot procedures as described in Example 2 with different water grains per gallon (GPG). The enzyme used in Table 9 is Lipase A. When the enzyme concentration is the same, but the water grain is different, Table 9 shows that the change in ghee removal is minimal for the TopUp procedure (TopUp: 62.6−66.7=4.1), but more substantial for the OnePot procedures (OnePot: 32.8−43.4=10.6).

TABLE 9

| | | Surf | Enzyme | | |
|---|---|---|---|---|---|
| Proce- | | Conc | Conc | | Water |
| dure | Surfactant | (ppm) | (ppm) | % SR | (GPG) |
| TopUp | C₁₀-C₁₂ alkylpolypentoside | 600 | 0 | 30.6 | DI |
| TopUp | C₁₀-C₁₂ alkylpolypentoside | 600 | 25 | 62.6 | DI |
| TopUp | C₁₀-C₁₂ alkylpolypentoside | 600 | 25 | 65.3 | 0 |
| TopUp | C₁₀-C₁₂ alkylpolypentoside | 600 | 25 | 66.7 | 5 |
| TopUp | C₁₀-C₁₂ alkylpolypentoside | 600 | 150 | 67.5 | DI |
| OnePot | C₁₀-C₁₂ alkylpolypentoside | 600 | 0 | 34.9 | DI |
| OnePot | C₁₀-C₁₂ alkylpolypentoside | 600 | 25 | 32.8 | DI |
| OnePot | C₁₀-C₁₂ alkylpolypentoside | 600 | 25 | 41.1 | 0 |
| OnePot | C₁₀-C₁₂ alkylpolypentoside | 600 | 25 | 43.4 | 5 |
| OnePot | C₁₀-C₁₂ alkylpolypentoside | 600 | 150 | 34.5 | DI |

Table 10 uses 4 different surfactants (an alkylpolypentoside, a C₈-C₁₆ alkylpolyglycoside, lauryl dimethylamine oxide, and an EO PO block copolymer) at the same concentration in the two different procedures as described in Example 2. Table 10 utilizes 3 different lipase enzymes at the same concentration.

TABLE 10

| | | Surf | | Enzyme | |
|---|---|---|---|---|---|
| Proce- | | Conc | | Conc | % |
| dure | Surfactant | (ppm) | Enzyme | (ppm) | SR |
| TopUp | C₁₀-C₁₂ | 600 | Lipase B | 150 | 42.1 |
| | alkylpolypentoside | | | | |
| TopUp | C₁₀-C₁₂ | 600 | Lipase C | 150 | 62.5 |
| | alkylpolypentoside | | | | |
| TopUp | C₁₀-C₁₂ | 600 | Lipase A | 150 | 65.9 |
| | alkylpolypentoside | | | | |
| TopUp | alkylpolyglycoside | 600 | Lipase B | 150 | 36.7 |
| TopUp | alkylpolyglycoside | 600 | Lipase C | 150 | 49.8 |
| TopUp | alkylpolyglycoside | 600 | Lipase A | 150 | 54.6 |
| TopUp | lauryl dimethylamine | 600 | Lipase B | 150 | 19.5 |
| | oxide | | | | |
| TopUp | lauryl dimethylamine | 600 | Lipase C | 150 | 40.4 |
| | oxide | | | | |
| TopUp | lauryl dimethylamine | 600 | Lipase A | 150 | 54.4 |
| | oxide | | | | |
| TopUp | EO PO block copolymer | 600 | Lipase B | 150 | 77.0 |
| TopUp | EO PO block copolymer | 600 | Lipase C | 150 | 75.2 |
| TopUp | EO PO block copolymer | 600 | Lipase A | 150 | 72.8 |
| OnePot | C₁₀-C₁₂ | 600 | Lipase B | 150 | 37.6 |
| | alkylpolypentoside | | | | |
| OnePot | C₁₀-C₁₂ | 600 | Lipase C | 150 | 49.6 |
| | alkylpolypentoside | | | | |
| OnePot | C₁₀-C₁₂ | 600 | Lipase C | 150 | 36.0 |
| | alkylpolypentoside | | | | |
| OnePot | alkylpolyglycoside | 600 | Lipase B | 150 | 26.1 |
| OnePot | alkylpolyglycoside | 600 | Lipase C | 150 | 38.9 |
| OnePot | alkylpolyglycoside | 600 | Lipase C | 150 | 40.0 |
| OnePot | lauryl dimethylamine | 600 | Lipase B | 150 | 21.9 |
| | oxide | | | | |
| OnePot | lauryl dimethylamine | 600 | Lipase C | 150 | 21.5 |
| | oxide | | | | |
| OnePot | lauryl dimethylamine | 600 | Lipase C | 150 | 30.1 |
| | oxide | | | | |
| OnePot | EO PO block copolymer | 600 | Lipase B | 150 | 71.4 |
| OnePot | EO PO block copolymer | 600 | Lipase C | 150 | 70.5 |
| OnePot | EO PO block copolymer | 600 | Lipase C | 150 | 78.0 |

As can be seen in Table 10, all of the lipases performed better using the TopUp procedure when compared with the standard OnePot procedure. Additionally, while Lipase A and C tended to perform better than Lipase B when paired with alkyl poylglycosides and alkylpolypentosides, it should be noted that Lipase B performed better with EO/PO block copolymers. Thus, it can be seen that regardless of the lipase employed, the use of TopUp procedure provides a synergistic result compared to the OnePot procedure.

Table 11 uses the 2 different surfactants (a C₁₂-C₁₄ alkylpolypentoside and a C₈-C₁₆ alkylpolyglycoside) at 4 different concentrations (600, 300, 150, and 75 ppm), the same enzyme, and the same enzyme concentration in the two different procedures as described in Example 2. Table 11 shows that the surfactants performed best at higher concentrations than the enzyme in either of the procedures.

TABLE 11

| | | Surf | | Enzyme | |
|---|---|---|---|---|---|
| | | Conc | | Conc | % |
| Procedure | Surfactant | (ppm) | Enzyme | (ppm) | SR |
| TopUp | C₁₀-C₁₂ | 600 | Lipase C | 150 | 83.02 |
| | alkylpolypentoside | | | | |
| TopUp | C₁₀-C₁₂ | 300 | Lipase C | 150 | 69.05 |
| | alkylpolypentoside | | | | |
| TopUp | C₁₀-C₁₂ | 150 | Lipase C | 150 | 66.75 |
| | alkylpolypentoside | | | | |
| TopUp | C₁₀-C₁₂ | 75 | Lipase C | 150 | 55.98 |
| | alkylpolypentoside | | | | |
| TopUp | alkylpolyglycoside | 600 | Lipase C | 150 | 66.64 |
| TopUp | alkylpolyglycoside | 300 | Lipase C | 150 | 63.30 |
| TopUp | alkylpolyglycoside | 150 | Lipase C | 150 | 48.53 |
| TopUp | alkylpolyglycoside | 75 | Lipase C | 150 | 50.04 |
| OnePot | C₁₀-C₁₂ | 600 | Lipase C | 150 | 56.75 |
| | alkylpolypentoside | | | | |
| OnePot | C₁₀-C₁₂ | 300 | Lipase C | 150 | 48.97 |
| | alkylpolypentoside | | | | |
| OnePot | C₁₀-C₁₂ | 150 | Lipase C | 150 | 46.42 |
| | alkylpolypentoside | | | | |
| OnePot | C₁₀-C₁₂ | 75 | Lipase C | 150 | 46.00 |
| | alkylpolypentoside | | | | |
| OnePot | alkylpolyglycoside | 600 | Lipase C | 150 | 37.72 |
| OnePot | alkylpolyglycoside | 300 | Lipase C | 150 | 41.32 |
| OnePot | alkylpolyglycoside | 150 | Lipase C | 150 | 36.13 |
| OnePot | alkylpolyglycoside | 75 | Lipase C | 150 | 36.72 |

### Example 5

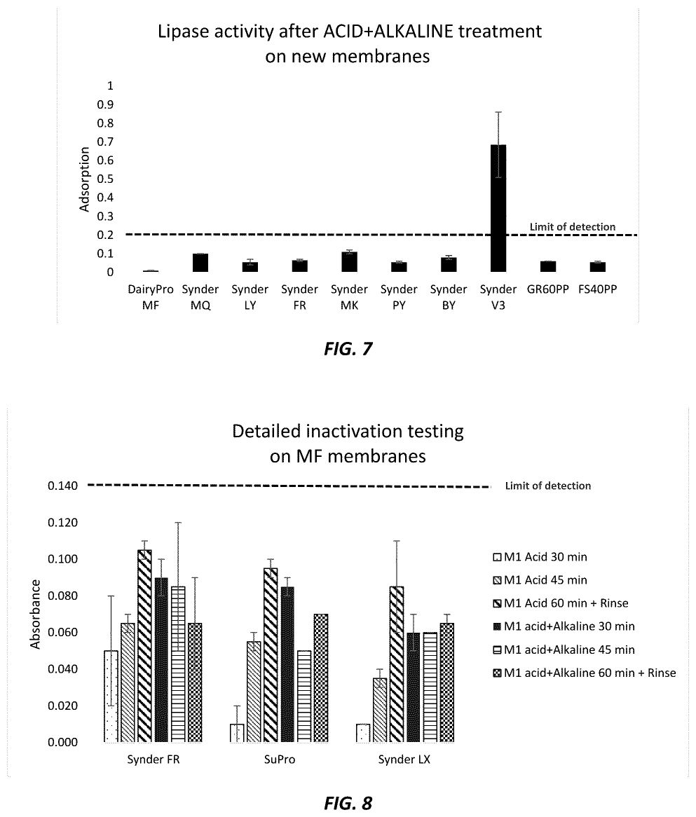

The exemplary products and procedures of the above examples were tested on 10 different microfiltration and ultrafiltration membranes (Dairy Pro MF, Synder MQ, Synder LY, Synder FR, Synder MK, Synder PY, Synder BY, Synder V3, GR60PP, and FS40PP). The membranes were additionally washed with an optional alkaline treatment. The lipase adsorption of the membranes was then detected and plotted on a chart to compare the membranes, which is shown in FIG. 7. Most of the membranes tested are microfiltration membranes and have a molecular weight cut-off above the lipase size (approximately 30 kDa), except GR60PP. As FIG. 7 shows, after both the inactivation step and optional alkaline wash, the lipase activity is substantially below the limit of detection. One outlier, Synder V3 is a cationic membrane. A cationic membrane surface increases the surface interaction with the lipase, which makes the inactivation of the lipase more difficult than a membrane which is not cationic. These membranes are not generally used in food applications. Thus, the exemplary cleaning regimes and procedures of the above examples are highly effective in inactivating lipase and cleaning membranes. The combination of an additional alkaline step helps to reduce surfactant residues and has a positive contribution to the inactivation procedure.

Three microfiltration membranes (Synder FR, SuPro, and Synder LX) were additionally tested for absorbance of lipase after the exemplary cleaning regimes at 30, 45, and 60 minutes with and without the alkaline rinse. FIG. 8 shows the results of these tests. As can be seen in the chart of FIG. 8, all the exemplary cleaning regimes result in inactivation of the lipase, and remaining lipase being below the limit of detection.

### Example 6

Example cleaning regimes were tested at various dairy membrane plants. Three different examples were tested on filtration systems to assess the methods as applied to different systems.

### System A

The first filtration system included 4 loop ultrafiltration membranes producing WPC30. The membranes were about 1.5 years old at the time point of the CIP test, and system was compromised with denatured proteins and fat fouling. Data was accessed by consolidation Key Performance Indicators during the production phase. The tested clean-in-place concept was based on lipase, protease and APG surfactants.

Before the filtration system was initially cleaned with the Lipase based CIP concept, a fouling potential was observed. The baseline enzymatic clean-in-place regime showed significant fouling trend via an increased pressure increase over the membrane life. The lipase cleaning resulted in lower fouling trend slope of pressure normalized Permeate Flow around 200%. Also, with the lipolytic Cleaning in Place Top Up process we observed that the membranes' average retentate capacity was increased by 275%. The lipase cleaned-in-place can positively impact fouling behavior, an increase in membrane lifetime, lower pressure input, and results an overall higher pressure normalized Permeate Flow. The special cleaning with lipase confirmed results that increased the overall filtration capacity around 275% with only one cleaning-in-place. The effect on productivity stabilized minimum two weeks after the single lipase cleaning event was conducted.

### System B

The second dairy filtration system comprised a combined Reverse Osmosis/Nanofiltration membrane system, which processes WPC30 to WPC80. Cleaning performance of the TopUp accessed by the Clean Water Fluxes was taken after the CIP Phase. A clean-in-place concept was based on lipase, protease and APG surfactants.

In the acidic phase, a verified inactivation concept for lipase inactivation was performed. Production, clean water flux data, and cleaned-in-place analysis were investigated. The cleaned-in-place solution resulted in high chemical oxygen demand-load in during lipase recirculation phase and showed an increase of degraded fatty acids showing removal of fats. Moreover, the solution resulted a full lipase inactivation providing no off-taste issues in the product and saw no incompatibility maters in the membranes.

The dairy filtration system saw a significant flux increase (˜10%) especially on the nanofiltration loops. The dairy filtration system also saw significantly increased production capacity.

### System C

The third dairy filtration system comprised a 4 loop RO/ROP membrane system for treatment of wastewater. Cleaning performance of the TopUp accessed by the Clean Water Fluxes taken after the CIP Phase. The cleaning resulted in significant soil removal versus baseline and precleaning steps, and a significant increase in fluxes with every extra cleaning.

After switching back to the baseline regime, the dairy filtration system showed a significant flux decline. A further increase in flux was seen after an additional lipase clean. The dairy filtration system showed an improved cleaning efficiency while also having an increased productivity.

The disclosures being thus described, it will be obvious that the same may be varied in many ways. Such variations are not to be regarded as a departure from the spirit and scope of the disclosures and all such modifications are intended to be included within the scope of the following claims. The above specification provides a description of the manufacture and use of the disclosed compositions and methods. Since many embodiments can be made without departing from the spirit and scope of the disclosure, the invention resides in the claims.

What is claimed is:

1. A method of cleaning a dairy filtration membrane comprising:
(a) contacting the dairy filtration membrane with an enzyme composition, wherein the enzyme composition comprises a lipase and a buffer, wherein the pH of the enzyme composition is from about 7.5 to about 11.0;
(b) contacting the dairy filtration membrane with a surfactant composition, wherein the surfactant composition comprises an alkyl polyglucoside, an alkyl polypentoside, an amine oxide, an alcohol ethoxylate, an alkoxylated block copolymer, a sulfonated surfactant, or a mixture thereof; wherein the pH of the surfactant composition is from about 7.5 to about 11.0;
(c) rinsing the dairy filtration membrane;
(d) contacting the dairy filtration membrane with an acidic composition, wherein the acidic composition comprises an acid, an acidic anionic surfactant, or a mixture thereof; wherein the pH of the acidic composition is about 2.5 or lower; wherein the acidic composition deactivates the lipase; and
(e) rinsing the dairy filtration membrane.

2. The method of claim 1, wherein the lipase comprises a lipase derived from a Pseudomonas or a Humicola.

3. The method of claim 1, wherein the enzyme composition further comprises a protease.

4. The method of claim 3, wherein the protease comprises a protease derived from Bacillus subtilis, Bacillus licheniformis and Streptomyces griseus.

5. The method of claim 1, wherein the buffer comprises an alkali metal hydroxide, alkali metal carbonate, sodium bicarbonate, or a mixture thereof.

6. The method of claim 1, wherein the enzyme composition comprises a chelant; and
wherein the chelant comprises an aminocarboxylic acid or salt thereof, sodium tripolyphosphate, citric acid or a salt thereof, or a mixture thereof.

7. The method of claim 1, wherein the acidic composition comprises a linear alkyl sulfonate, linear alkyl sulfate, a branched alkyl sulfonate, a branched alkyl sulfate, a substituted aromatic sulfonate, an unsubstituted aromatic sulfonate, an organic acid, nitric acid, phosphoric acid, methane sulfonic acid, or mixture thereof.

8. The method of claim 1, wherein the surfactant composition comprises one or more of a C₅, C₁₂-C₁₄ alkylpolypentoside, a C₈-C₁₆ alkylpolyglycoside, an EO PO block copolymer, nonylphenol ethoxylate, lauryl dimethylamine oxide, linear alkylbenzene sulfonate, 2-ethyl-hexyl alkoxylate, a C₆ alkyl polyglycoside, a secondary n-alkyl sulfonate, sodium lauryl sulfonate; sodium C₁₂₋₁₄ alcohol sulfate, or a mixture thereof.

9. The method of claim 1, further comprising
(f) contacting the dairy filtration membrane with an alkaline composition; wherein the alkaline composition has a pH from about 9.5 to about 12.0; and
(g) rinsing the dairy filtration membrane.

10. The method of claim 9, wherein the alkaline composition comprises an alkali metal hydroxide, alkali metal carbonate, an alkali metal silicate, an organic alkalinity source, or mixture thereof.

11. The method of claim 1, further comprising the step of pre-rinsing the dairy filtration membrane before step (a).

12. The method of claim 1, further comprising, before step (a), the step of contacting the dairy filtration membrane with a membrane detergent composition and subsequently rinsing the dairy filtration membrane.

13. The method of claim 1, wherein the dairy filtration membrane comprises a microfiltration membrane, an ultrafiltration membrane, nanofiltration membrane, and/or a reverse osmosis membrane.

14. The method of claim 2, wherein the lipase comprises a variant of the wild-type Thermomyces lanuginosus lipase.

15. The method of claim 14, wherein the lipase has at least 90% sequence identity to SEQ ID NO 1.

16. A method of cleaning a dairy filtration membrane comprising:
(a) contacting the dairy filtration membrane with an enzyme composition, wherein the enzyme composition comprises a lipase, a protease, and a buffer, wherein the pH of the enzyme composition is from about 8.0 to about 10.5; wherein the dairy filtration membrane comprises a microfiltration membrane, an ultrafiltration membrane, nanofiltration membrane, and/or a reverse osmosis membrane;
(b) contacting the dairy filtration membrane with a surfactant composition, wherein the surfactant composition comprises an alkyl polyglucoside, an alkyl polypentoside, an amine oxide, an alcohol ethoxylate, an alkoxylated block copolymer, a sulfonated surfactant, or a mixture thereof; wherein the pH of the surfactant composition is from about 8.0 to about 10.5;
(c) rinsing the dairy filtration membrane;
(d) contacting the dairy filtration membrane with an acidic composition, wherein the acidic composition comprises a linear alkyl sulfonate, a branched alkyl sulfonate, a substituted aromatic sulfonate, an unsubstituted aromatic sulfonate, an organic acid, nitric acid, phosphoric acid, methanesulfonic acid, or mixture thereof, or a mixture thereof; wherein the pH of the acidic composition is about 2.5 or lower; wherein the acidic composition deactivates the lipase; and
(e) rinsing the dairy filtration membrane.

17. The method of claim 16, further comprising:
(f) contacting the dairy filtration membrane with an alkaline composition; wherein the alkaline composition comprises an alkali metal hydroxide, alkali metal carbonate, an alkali metal silicate, an organic alkalinity source, or mixture thereof; wherein the alkaline composition has a pH from about 10.0 to about 12.0; and
(g) rinsing the dairy filtration membrane.

18. The method of claim 16, wherein the lipase comprises a lipase derived from a Pseudomonas or a Humicola; wherein the protease comprises a protease derived from Bacillus subtilis, Bacillus licheniformis and Streptomyces griseus; wherein the buffer comprises an alkali metal carbonate, sodium bicarbonate, or a mixture thereof; and wherein the enzyme composition further comprises a chelant; wherein the chelant comprises an aminocarboxylic acid or salt thereof, sodium tripolyphosphate, citric acid or a salt thereof, or a mixture thereof.

19. The method of claim 16, wherein the surfactant composition further comprises a protease; wherein the protease comprises a protease derived from Bacillus subtilis, Bacillus licheniformis and Streptomyces griseus.

20. The method of claim 16, wherein the surfactant composition comprises one or more of a C₅, C₁₂-C₁₄ alkylpolypentoside, a C₈-C₁₆ alkylpolyglycoside, an EO PO block copolymer, nonylphenol ethoxylate, lauryl dimethylamine oxide, linear alkylbenzene sulfonate, 2-ethyl-hexyl alkoxylate, a C₆ alkyl polyglycoside, a secondary n-alkyl sulfonate, sodium lauryl sulfonate; sodium C₁₂₋₁₄ alcohol sulfate, or a mixture thereof.
